(19) United States
(12) Patent Application Publication
(10) Pub. No.: US 2026/0096735 A1
(43) Pub. Date: Apr. 9, 2026

(54) DEVICE, SYSTEM, AND METHOD FOR DETERMINING ARTERIAL AND VENOUS BLOOD PRESSURES

(71) Applicant: UNIVERSITY OF PITTSBURGH-OF THE COMMONWEALTH SYSTEM OF HIGHER EDUCATION, Pittsburgh, PA (US)

(72) Inventors: Cederick LANDRY, Pittsburgh, PA (US); Vishaal DHAMOTHARAN, Pittsburgh, PA (US); Mark FREITHALER, Pittsburgh, PA (US); Alisse HAUSPURG-JANICKI, Pittsburgh, PA (US); Aman MAHAJAN, Pittsburgh, PA (US); Sanjeev Govinddas SHROFF, Pittsburgh, PA (US); Shipeng WANG, Pittsburgh, PA (US); Ramakrishna MUKKAMALA, Sewickley, PA (US)

(73) Assignee: UNIVERSITY OF PITTSBURGH-OF THE COMMONWEALTH SYSTEM OF HIGHER EDUCATION, Pittsburgh, PA (US)

(21) Appl. No.: 19/417,682

(22) Filed: Dec. 12, 2025

Related U.S. Application Data
(63) Continuation of application No. PCT/US2024/039366, filed on Jul. 24, 2024.
(60) Provisional application No. 63/515,239, filed on Jul. 24, 2023.

(51) Int. Cl. A61B 5/022; A61B 5/00; A61B 5/02; A61B 90/00

(52) U.S. Cl. CPC A61B 5/02225; A61B 5/02007; A61B 5/6824; A61B 5/6826; A61B 5/7239; A61B 5/7271; A61B 5/7405; A61B 5/742; A61B 5/7475; A61B 90/06; A61B 2090/064

(57) ABSTRACT

Device, method, and system for cuffless blood pressure (BP) measurement are provided. The device, method, and system include the use of a device with a photoplethysmography (PPG)-force sensor unit, an automatic cuff device, or an oscillometric device. The disclosed subject matter is provided to improve the accuracy of the cuffless blood pressure monitoring, the accuracy of automatic arm cuff/cuffless devices, and increase signal quality of measuring devices. The disclosed subject matter can be used in conjunction with smartphones, wearables, or wrist cuffs.

### CROSS-REFERENCE TO RELATED APPLICATIONS

This application is a continuation of International Application No. PCT/US2024/039366, filed on Jul. 24, 2024, which claims priority to U.S. Provisional Application Ser. No. 63/515,239, filed on Jul. 24, 2023, which are incorporated by reference herein in their entireties.

### STATEMENT REGARDING FEDERALLY SPONSORED RESEARCH OR DEVELOPMENT

This invention was made with government support under grant nos. HL146470 and HL163691 awarded by the National Institutes of Health. The government has certain rights in the invention.

### FIELD

The presently disclosed subject matter relates to devices, systems, and methods for measuring blood pressure. The disclosed device and systems can determine the arterial and venous blood pressures of a subject.

### BACKGROUND

Oscillometry is the blood pressure (BP) measurement principle employed by most automatic cuff devices. This principle exploits the sigmoidal blood volume-transmural pressure relationship of arteries (where transmural pressure=internal BP-external pressure of the artery). The basic idea is to slowly vary the external pressure of the artery to first increase and then decrease the blood volume oscillation amplitude and thereafter apply an algorithm to the observed blood volume oscillation amplitude versus external pressure function (“oscillogram”) to compute BP. As shown in FIG. 1 (prior art), conventional devices slowly inflate or deflate a cuff placed over the brachial artery to vary its external pressure and measure the pressure waveform inside the cuff to obtain both the external pressure via lowpass filtering and the variable-amplitude blood volume oscillations via highpass filtering and apply population average algorithms to compute systolic/mean/diastolic BP from the oscillogram. These algorithms are thus less accurate in atypical people, such as those with wide pulse pressure due to large artery stiffening.

The oscillometric finger pressing method extends the automatic cuff device principle for cuffless BP monitoring via a smartphone [1]. FIG. 2 illustrate the concept. The user serves as the actuator (instead of the cuff) by pressing their fingertip against the phone (nominally held at heart level) to steadily increase the external pressure of the underlying artery. The phone, embedded with photoplethysmography (PPG) and force transducers, serves as the sensor (rather than the cuff device) to measure the resulting variable blood volume oscillations and applied finger pressure. The phone also provides visual feedback to guide the amount of finger pressure applied over time and employs an algorithm to compute BP from the measurements, like a cuff device. Accurate computation of BP from the finger oscillometric measurements is a key challenge due to physiological differences between finger and arm arteries and BP levels.

Hence, there is a need for improved techniques for accurate oscillometric BP computation.

### SUMMARY OF THE DISCLOSED SUBJECT MATTER

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter includes a device, method, and system for blood pressure monitoring.

The disclosed subject matter provides a method for determining a blood pressure value of a user using a device with a photoplethysmography (PPG)-force sensor unit. The method can include providing visual or audio instructions to the user with the device, measuring PPG oscillations of the finger and the finger pressures with the PPG-force sensor unit, and computing a finger artery viscoelastic marker from the PPG oscillations. In non-limiting embodiments, the instructions can instruct the user to position a finger on the PPG-force sensor unit and to press the finger on the PPG-force sensor unit at varying finger pressures. The method can further include extracting at least one additional feature from an oscillation height versus finger pressure or a PPG oscillation shape versus finger pressure function, computing a blood pressure value using the finger artery viscoelastic marker and the at least one additional feature, and outputting the blood pressure value on a graphical user interface of the device or sending the blood pressure value to a database repository.

In certain embodiments the finger artery viscoelastic marker can include an average of a normalized PPG waveform.

In certain embodiments, the finger artery viscoelastic marker can include a root-mean-square of a normalized PPG waveform.

In certain embodiments the at least one additional feature can include a width or peak position of the height oscillation vs finger pressure function.

In certain embodiments, the at least one additional feature can be extracted from an oscillation width vs finger pressure function.

The disclosed subject matter also provides a method for determining a blood pressure value of a user using an automatic cuff device. The method can include measuring an oscillometric cuff pressure waveform with the automatic cuff device, constructing a height oscillation versus a cuff pressure function from the oscillometric cuff pressure waveform, constructing an area oscillation versus the cuff pressure function of the oscillometric cuff pressure waveform, and fitting a single mathematical model for the height and the area functions to the measured height oscillation versus the cuff pressure function and the measured area oscillation versus the cuff pressure function, respectively, in an optimal sense to estimate model parameters. In non-limiting embodiments, the parameters of the single mathematical model can include systolic blood pressure, diastolic blood pressure, and arterial compliance. Further, the method can include outputting the blood pressure value on a graphical user interface of the device or sending the blood pressure value to a database repository.

In certain embodiments, the mathematical model can be a sigmoidal blood volume-transmural pressure relation of arteries.

In certain embodiments, an arterial compliance curve, which can be a derivative of the sigmoidal relation, is defined by an asymmetric exponential function. In certain embodiments, an arterial compliance curve, which can be a derivative of the sigmoidal relation, is defined by an asymmetric exponential-linear function.

In certain embodiments, the mathematical model of the area function can use a triangular pulse train to represent a blood pressure waveform input.

In certain embodiments, the model can be fitted over a higher cuff pressure range. In non-limiting embodiments, the pressure-volume relation of a cuff transducer can be more linear than a pressure-volume relation obtained at a lower cuff pressure range.

The disclosed subject matter also provides a method for determining a venous pressure value of a user using an oscillometric device on a wrist or a hand. The method can include positioning the device lower than a heart level of the user, measuring or approximating a vertical distance between the device and a heart of the user, and measuring a blood volume and an applied external pressure of an artery at varying external pressure. The method can further include detecting an external pressure at a venous pressure marker from a measured blood volume vs external pressure function, subtracting a value from the external pressure at the venous pressure marker based on the vertical distance between the device and the heart to compute a venous pressure, and outputting the venous pressure value on a graphical user interface of the device or sending the venous pressure value to a database repository.

In certain embodiments, the device can be an automatic wrist cuff device.

In certain embodiments, the device can be an oscillometric finger pressing device. In non-limiting embodiments, the device can be held with the hand fully lowered.

In certain embodiments, the venous pressure can be detected from a peak position in blood volume oscillation vs external pressure function that can be lower than arterial pressure levels.

In certain embodiments, the venous pressure can be detected from a DC PPG measurement of the blood volume.

In certain embodiments, the disclosed subject matter provides a system for determining a blood pressure value of a user. The system can include a photoplethysmography (PPG)-force sensor and a processor. In non-limiting embodiments, the processor can be configured to provide visual or audio instructions to the user with the system, measure PPG oscillations of the finger and the finger pressures with the PPG-force sensor unit, compute a finger artery viscoelastic marker from the PPG oscillations, extract at least one additional feature from a PPG oscillation height versus finger pressure or a PPG oscillation shape versus finger pressure function, compute a blood pressure value using the finger artery viscoelastic marker and the at least one additional feature, and output the blood pressure value on a graphical user interface of the system or send the blood pressure value to a database repository. In non-limiting embodiments, the instructions can instruct the user to position a finger on a PPG-force sensor unit and to press the finger on a PPG-force sensor unit at varying finger pressures.

In certain embodiments, the disclosed subject matter provides an automatic cuff system for determining a blood pressure value of a user. The automatic cuff system can include a processor. The processor can be configured to measure an oscillometric cuff pressure waveform with the automatic cuff device, construct a height oscillogram versus a cuff pressure function from the oscillometric cuff pressure waveform, construct an area oscillogram versus the cuff pressure function of the oscillometric cuff pressure waveform, fit a single mathematical model for the height oscillogram and the area oscillogram to measured height and area oscillograms in an optimal sense to estimate model parameters, and output a blood pressure value on a graphical user interface of the device or send the blood pressure value to a database repository. In non-limiting embodiments, parameters of the single mathematical model can include systolic blood pressure, diastolic blood pressure, and arterial compliance.

In certain embodiments, the disclosed subject matter provides a system for determining a venous pressure value of a user. The disclosed system can include an oscillometric device on a wrist or a hand and a processor. In non-limiting embodiments, the processor can be configured to provide visual or audio instructions to the user to position the device lower than a heart level of the user, measure or approximate a vertical distance between the device and a heart of the user, measure a blood volume and an applied external pressure onto the wrist or hand at varying external pressure, detect an external pressure at a venous pressure marker from a measured blood volume vs external pressure function, subtract a value from the external pressure at the venous pressure marker based on the vertical distance between the device and the heart to compute a venous pressure, and output the venous pressure value on a graphical user interface of the device or send the venous pressure value to a database repository.

It is to be understood that both the foregoing general description and the following detailed description and drawings are examples and are provided for the purpose of illustration and not intended to limit the scope of the disclosed subject matter in any manner.

The accompanying drawings, which are incorporated in and constitute part of this specification, are included to illustrate and provide a further understanding of the devices of the disclosed subject matter. Together with the description, the drawings serve to explain the principles of the disclosed subject matter.

### BRIEF DESCRIPTION OF THE DRAWINGS

The subject matter of the application will be more readily understood from the following detailed description when read in conjunction with the accompanying drawings in which:

FIG. 1 (prior art) is a diagram illustrating the oscillometric blood pressure measurement principle employed by most automatic cuff devices.

FIG. 2 provides diagrams illustrating the oscillometric finger pressing method for cuffless and calibration-free monitoring of blood pressure (BP) via readily available smartphones.

FIG. 3 is a diagram illustrating the benchtop system for collecting oscillometric finger pressing data (top right panel) with a custom device and finger BP waveform data with a commercial volume-clamp finger cuff device.

FIG. 4 is a diagram illustrating the input-output data during oscillometric finger pressing used to identify nonlinear viscoelastic and elastic models.

FIGS. 5A-5C provide diagrams illustrating the nonlinear dynamic models for identification to quantify nonlinear viscoelasticity during oscillometric finger pressing. FIG. 5A shows Hammerstein viscoelastic model. FIG. 5B shows Wiener viscoelastic model. FIG. 5C shows Elastic model.

FIGS. 6A-6E provide graphs showing an average of fitting error and parameter estimates of the nonlinear viscoelastic and elastic models. FIG. 6A shows root-mean-square-error of the estimated PPG_{ac} waveform (RMSE) in percent. FIG. 6B shows root-mean-square-error of the constructed oscillogram (RMSEₒ) in percent. FIGS. 6C, 6D, and 6E shows b parameter (6C), c parameter (6D), and d parameter (6E) in terms of transfer function cutoff frequency.

FIGS. 7A-7D provide graphs showing the sensitivity of the derivative algorithm for oscillometric BP computation to the transfer function cutoff frequency of the Wiener model. FIG. 7A shows the systolic blood pressure (SP) estimation errors as a function of pulse pressure (PP) for different transfer function cutoff frequencies. FIG. 7B shows that the b parameter only slightly affects the SP estimation error. FIGS. 7C and 7D show the diastolic blood pressure (DP) estimation errors via Pₘₐₓₛₗₒₚₑ as a function of PP for different transfer function cutoff frequencies and for different c parameters, respectively.

FIGS. 8A-8C provide graphs showing the mean value of the normalized PPG at maximum oscillation during finger pressing is inversely correlated with the transfer function cutoff frequency of the Wiener model. FIGS. 8A and 8B show two representative samples of PPGₘₑₐₙ along with the estimated transfer function cutoff frequency (8A: 1.66 Hz and 8B: 4.24 Hz). FIG. 8C shows overall correlation plot between the cutoff frequency and PPGₘₑₐₙ. r is Pearson correlation coefficient.

FIG. 9 is a diagram illustrating the deconvolution of the variable PPG oscillations during oscillometric finger pressing with an estimated transfer function to mathematically remove the viscoelastic effect.

FIG. 10 provides graphs showing the deconvolution of the maximum PPG oscillation during oscillometric finger pressing with an estimated transfer function to obtain the finger BP waveform shape (Finapres Nova is a finger BP waveform via the volume-clamp finger cuff device).

FIGS. 11A and 11B provide graphs showing Cuff pressure oscillations and dc cuff pressure as measured with an automatic arm cuff. The oscillation shape changes from narrow to wide as the cuff pressure decreases (11A). Representative plot of an area oscillogram and height oscillogram showing different peak positions (11B).

FIG. 12 provides graphs showing the mathematical model of oscillometry with height oscillogram formula where the arterial compliance curve (a derivative of the sigmoidal blood volume-transmural pressure relation) is defined by an (asymmetric) exponential function for simplicity.

FIG. 13 provides diagrams illustrating the approach to derive the formula for the area oscillogram.

FIG. 14 is a graph showing measured P_{Amax} VS PHmax. (N=158).

FIGS. 15A-15E provide graphs showing normalized-root-mean-squared-error (NRMSE) of fitting the O_{A}(Pₑ) and O_{H}(Pₑ) formulas to the measured area and height oscillograms (N=158) (15A), average resulting b and c parameter estimates (15B), a normalized-root-mean-squared-error (NRMSE) of fitting oscillations using invasive BP waveform input and triangular pulse input (15C), Wiener and Hammerstein models fit height oscillograms better than the purely elastic model (15D), and the area oscillogram model is not significantly affected by viscoelastic effects represented by Wiener and Hammerstein models (15E).

FIG. 16 is a graph showing Predicted P_{Amax} VS measured P_{Amax} (N=158).

FIG. 17 is a diagram illustrating the skewness of an oscillation defined as the ratio of left-to-right areas.

FIG. 18 is a diagram illustrating representative examples of oscillation skewness and kurtosis versus cuff pressure (skewness and kurtosis oscillograms).

FIG. 19 provides graphs and images showing oscillometric finger pressing with hands fully lowered to improve the signal-to-noise ratio of the oscillogram and thereby detect venous pressure.

FIGS. 20A-20D provide images, graphs, and diagrams related to an oscillometric finger pressing method for measuring venous pressure. FIG. 20A shows an example device performing the oscillometric finger pressing method for measuring venous pressure. FIG. 20B provides graphs showing example oscillometric measurements from a subject. FIG. 20C shows an exemplary automated algorithm to detect the low-pressure variations. FIG. 20D shows the mean venous pressure detected with this algorithm for each participant at each arm cuff pressure.

FIG. 21 is a diagram illustrating the DC PPG vs finger pressure during oscillometric finger pressing that can also be used to detect venous pressure (VP).

FIG. 22 provides graphs showing a derivative algorithm for oscillometric BP computation in model simulations.

FIG. 23 provides a graph showing an example of model-estimated and measured PPGac waveforms during finger pressing.

FIG. 24 provides a graph showing constructed oscillograms from the sample in FIG. 23.

FIG. 25 provides a graph showing simulated oscillograms via the model with median parameter values and SP=110 mmHg and DP=70 mmHg.

FIGS. 26A-26C provide graphs showing representative oscillograms constructed from the original PPGac waveform and the deconvolved PPGac waveform.

FIG. 26A provides a graph showing measurements from a participant. FIG. 26B provides a graph showing Wiener model simulations. FIG. 26C provides a graph showing Hammerstein model simulations.

FIG. 27 provides a diagram showing equivalence of the best-fitting Wiener model to a physical model of oscillometric finger pressing measurements.

FIG. 28 shows an illustrative method for determining a blood pressure value.

FIG. 29 shows another illustrative method for determining a blood pressure value.

FIG. 30 shows an illustrative method for determining the venous blood pressure.

### DETAILED DESCRIPTION

Reference will now be made in detail to embodiments of the disclosed subject matter, an example of which is illustrated in the accompanying drawings. The disclosed subject matter will be described in conjunction with a detailed description of the system.

The terms used in this specification generally have their ordinary meanings in the art, within the context of the disclosed subject matter, and in the specific context where each term is used. Certain terms are discussed below or elsewhere in the specification to provide additional guidance to the practitioner in describing the compositions and methods of the disclosed subject matter.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing,” and “comprising” are interchangeable, and one of the skills in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

A “user” or “subject” herein is a vertebrate, such as a human or non-human animal, for example, a mammal. Mammals include, but are not limited to, humans, primates, farm animals, sport animals, rodents, and pets.

Here, the disclosed subject matter discloses methods for more accurate oscillometric BP computation via automatic cuff devices and ubiquitous smartphones. The disclosed subject matter also extend the oscillometric principle to measure venous blood pressure with greater fidelity using these and other devices. While BP (arterial pressure) monitoring is useful for detecting and controlling hypertension and managing hypotension in hospitals, venous pressure monitoring could give an early indication of fluid overload in heart failure patients and postpartum women with hypertensive disorders of pregnancy and thereby help avert frequent and costly hospitalizations.

### BP Computation Methods: Accounting for Nonlinear Viscoelasticity of Finger Arteries

Oscillometric BP measurement is conventionally applied to the brachial artery. The brachial artery is large (e.g., compared to finger arteries) and can often be well approximated as purely elastic (i.e., the artery expands instantaneously with a step increase in pressure). Models of arm cuff oscillometry are indeed typically based on the purely elastic, sigmoidal blood volume-transmural pressure relationship of the brachial artery [2]. For convenience, the sigmoidal model has also been employed to develop algorithms to compute finger BP via the oscillometric finger pressing method [3]. However, smaller finger arteries can be substantially more viscoelastic (i.e., the artery expands slowly in response to a step increase in pressure). Experimental data show that finger arteries exhibit readily apparent viscoelasticity (e.g., hysteresis loops within a beat and during finger cuff inflation-deflation cycles) [4]. Finger artery viscoelasticity increases with smooth muscle contraction, which varies acutely over time and from person to person. Finger artery viscoelasticity could thus adversely impact the accuracy of BP computation algorithms, especially those built on a purely elastic artery assumption. Using the disclosed subject matter, system identification can be applied to data to find a quantitatively significant finger artery viscoelastic effect. The performed simulations using the identified model further show that the extent of viscoelasticity has impact on finger BP computation errors. Methods were conceived to correct for the viscoelastic effect and thereby compute finger BP more accurately via the oscillometric finger pressing method.

### Model Identification and Simulations

Oscillometric finger pressing and finger BP waveform data were collected, and nonlinear viscoelastic and elastic models were identified from (i.e., fitted to) these input-output data. Simulated oscillometric finger pressing data with the best-fitting model were collected to analyze the accuracy of popular BP computation algorithms.

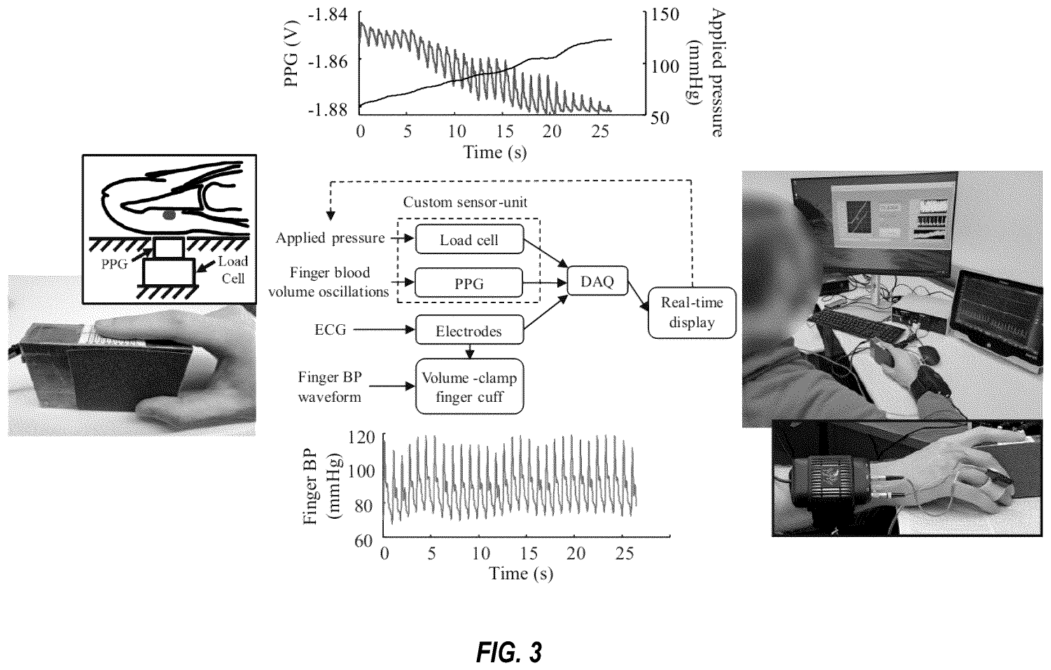

A benchtop system was used to collect the oscillometric finger pressing and finger BP waveform data from human volunteers under IRB approval. FIG. 3 illustrates the benchtop system. The finger BP waveform data were collected with a volume-clamp finger cuff device. This device is bulky and expensive but used in this basic investigation to quantify finger artery viscoelasticity.

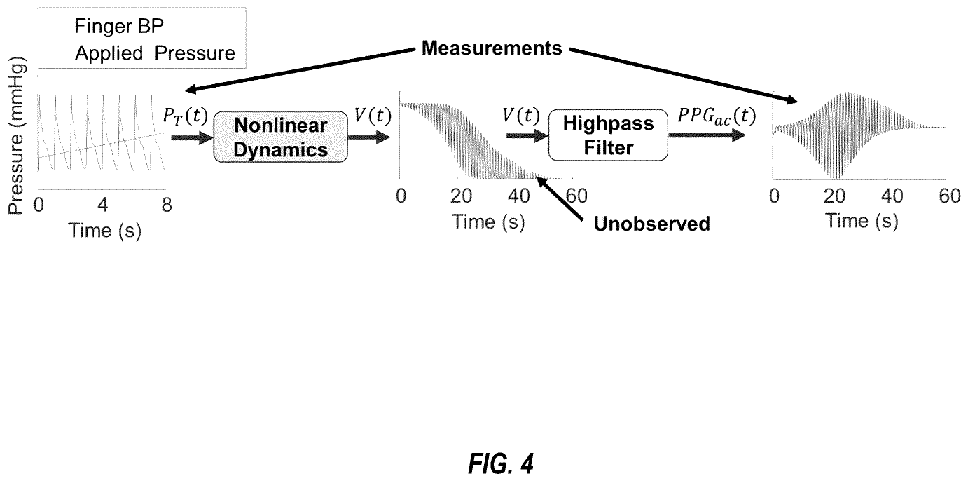

The nonlinear viscoelastic and elastic models relate the transmural pressure (P_{T}(t); finger BP waveform-applied finger pressure) to the blood volume in the artery (V(t)), as shown in FIG. 4. While absolute blood volume cannot be measured, photoplethysmography (PPG) can measure a waveform proportional to the arterial blood volume oscillations (ac signal).

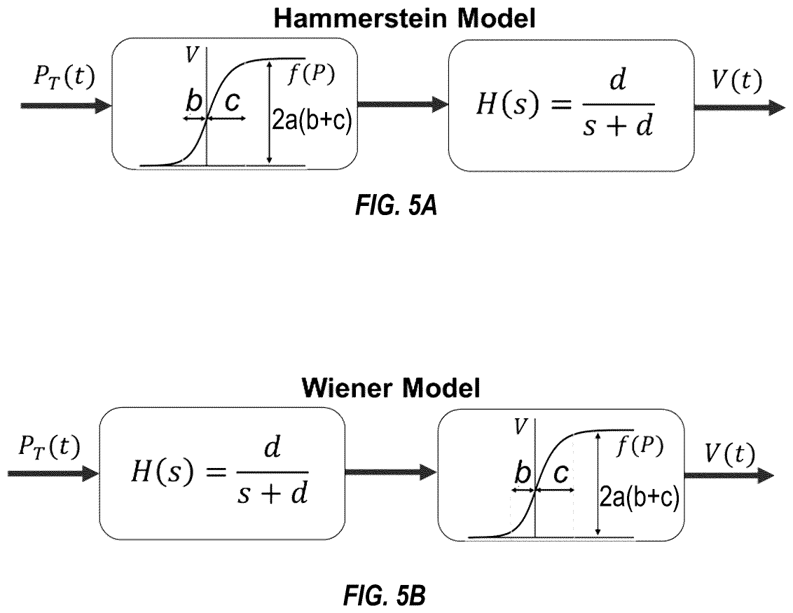

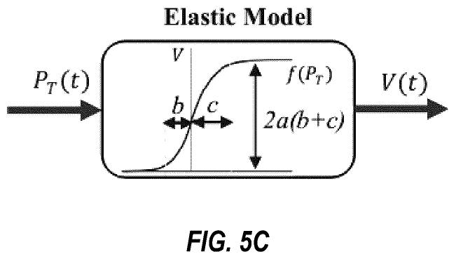

As shown in FIGS. 5A-5C, the data were specifically fitted to two standard nonlinear dynamic models-Hammerstein and Wiener models. Both models include static nonlinearity (sigmoidal exponential-linear function [2]) and linear dynamics (single pole, unity-gain transfer function). The nonlinear optimization was used to estimate the model parameters (a, b, c, and d) and determine the goodness-of-fit per individual. An elastic model (i.e., without the transfer function) was likewise identified.

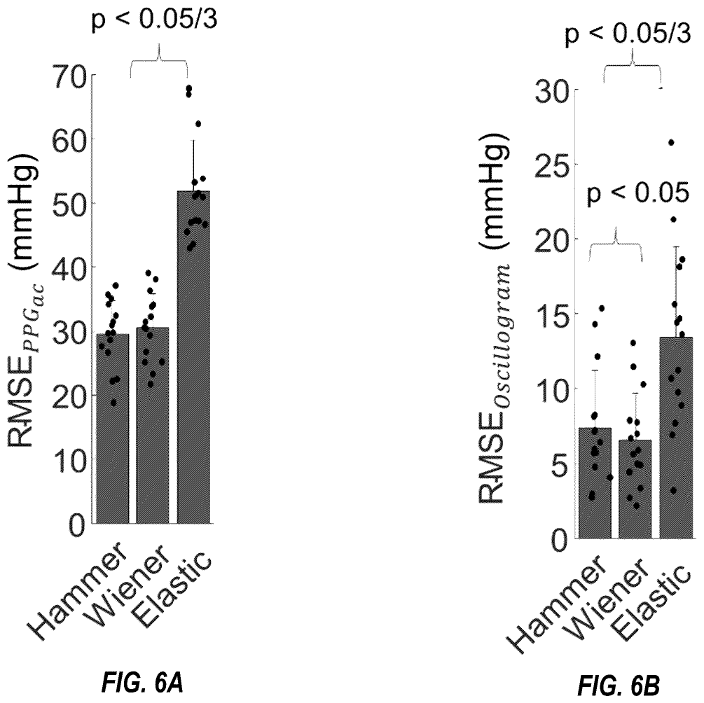

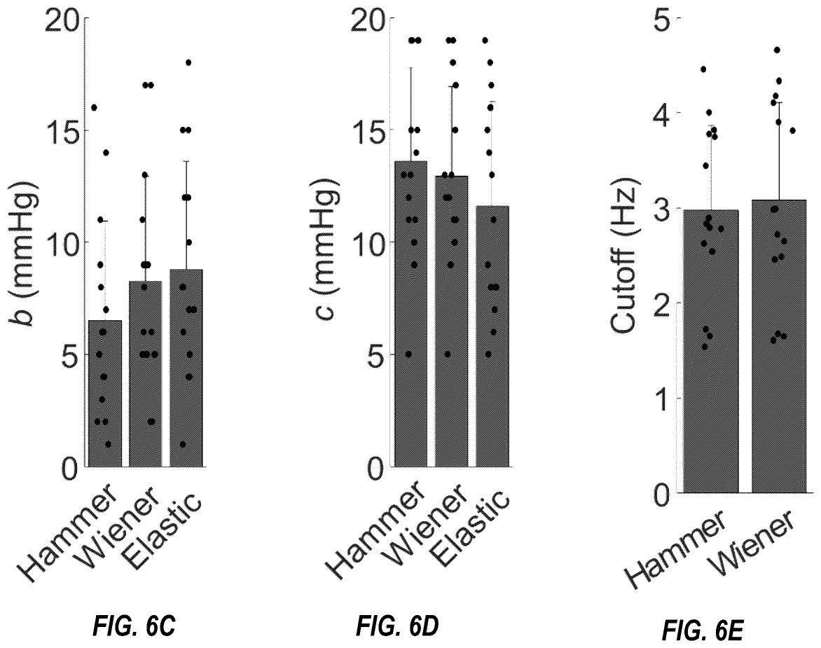

Viscoelasticity was quantitatively significant with the average fitting errors along with the parameter estimates shown in FIGS. 6A-6E (N=15). The Wiener model yielded a lower oscillogram (oscillation amplitude vs finger pressure) fitting error and was selected as the best model.

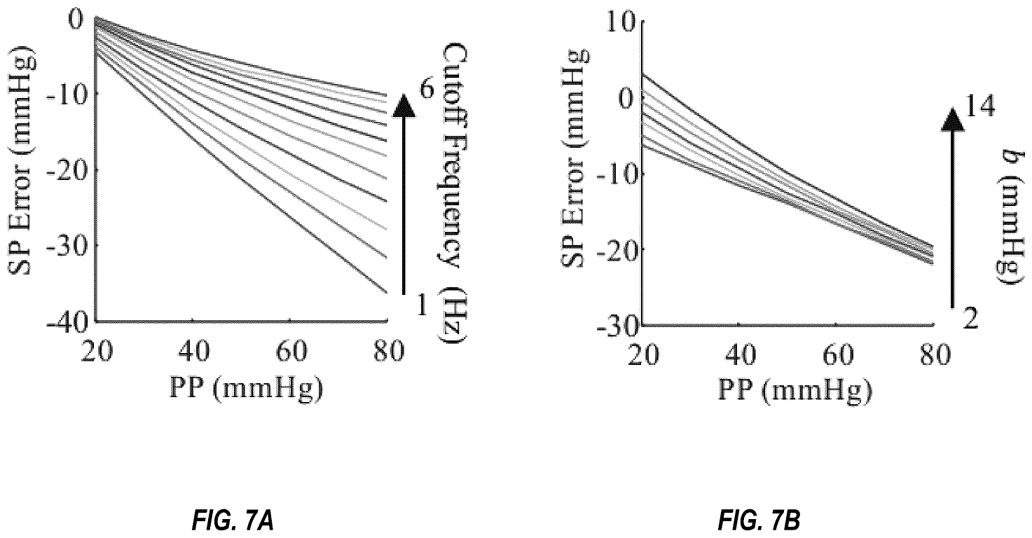

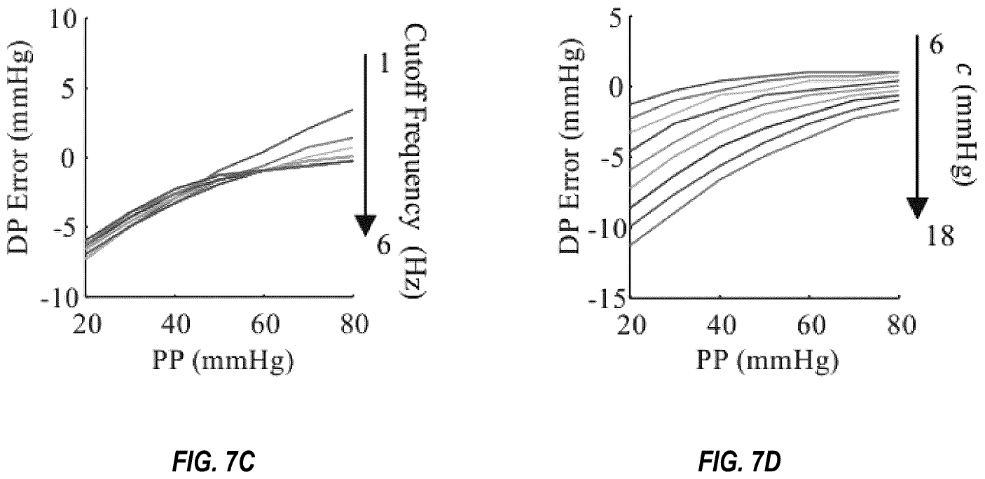

The Wiener model plus parametric sensitivity analysis was performed to simulate various sets of oscillometric finger pressing data. As shown in FIGS. 7A-7D, increasing viscoelasticity (as quantified by decreasing transfer function cutoff frequency) led to mild misestimation of diastolic BP (DP) but large underestimation of systolic BP (SP) when using the popular derivative algorithm for oscillometric BP computation [2]. In sum, the extent of viscoelasticity can have an important impact on finger BP computation errors.

### Correction for Finger Artery Viscoelasticity

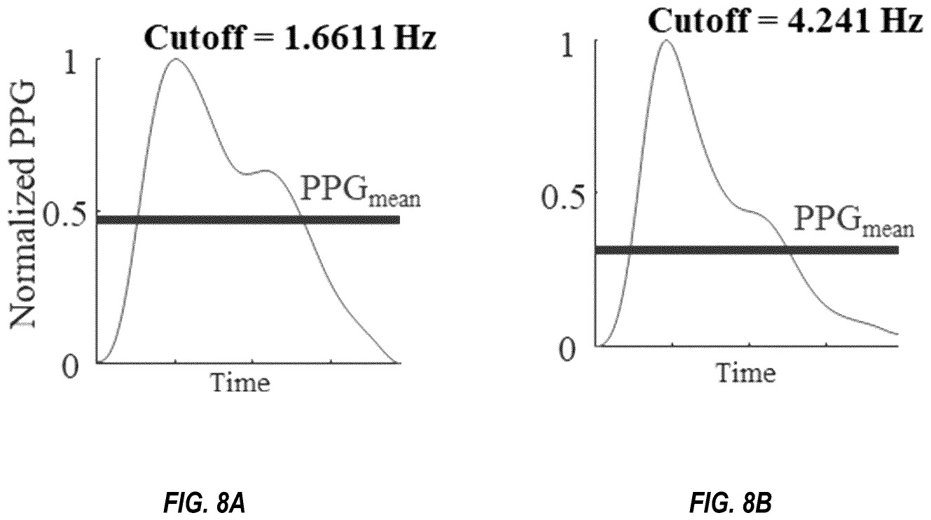

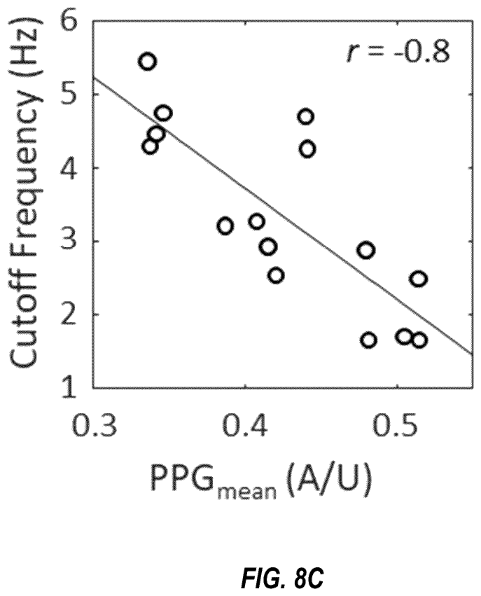

Markers to infer the extent of finger artery viscoelasticity (quantified again by the cutoff frequency of the transfer function) were identified from the oscillometric finger pressing data. Increasing finger viscoelasticity essentially means greater damping/lowpass filtering of the input finger BP waveform. The mean of the normalized PPG signal at maximum oscillation (where the artery is most linear) indeed showed a good negative correlation with the reference transfer function cutoff frequency determined using the finger BP waveform data, as shown in FIGS. 8A-8C. Although only the mean PPG is shown, any metric of the bulkiness of the PPG signal could be used including the root-mean square of the signal. Using a few beats (e.g., 1-3) before and after the PPG at maximum oscillation and averaging could lead to a more robust estimation of finger artery viscoelasticity.

These and other markers could be used as input to a BP computation algorithm to compensate for the viscoelasticity. For instance, they could be used directly as input (along with other features extracted from the oscillometric finger pressing data) to a machine learning model to compute BP. Another example is to use the model to compensate for the error of popular algorithms (e.g., derivative algorithm). For instance, for a given algorithm, a mapping between BP error, PP estimate, and cutoff frequency (see FIG. 7) can be created using training data comprising oscillometric finger pressing data and reference cuff BP. Then for a given BP measurement, the PP estimate is known, and the cutoff frequency can be estimated (e.g., from the mean of the normalized PPG signal). In this way, the expected BP error can be computed and added to the BP measurement to correct for finger artery viscoelasticity.

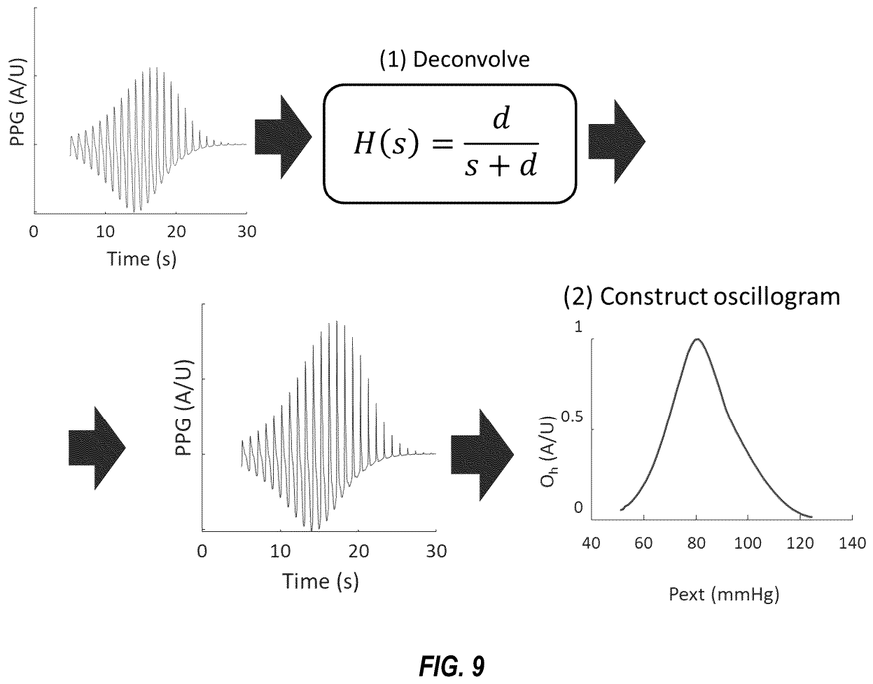

The viscoelastic effect could also be mathematically removed from the oscillometric finger pressing data to arrive at “undamped/purely elastic oscillograms”—where popular oscillometric BP computation algorithms are more accurate. For instance, undamped oscillograms can be retrieved by deconvolving the variable PPG oscillations during finger pressing with the transfer function comprising the estimated cutoff frequency, as shown in FIG. 9. Standard oscillometric algorithms can then be applied to these undamped oscillograms to compute finger BP.

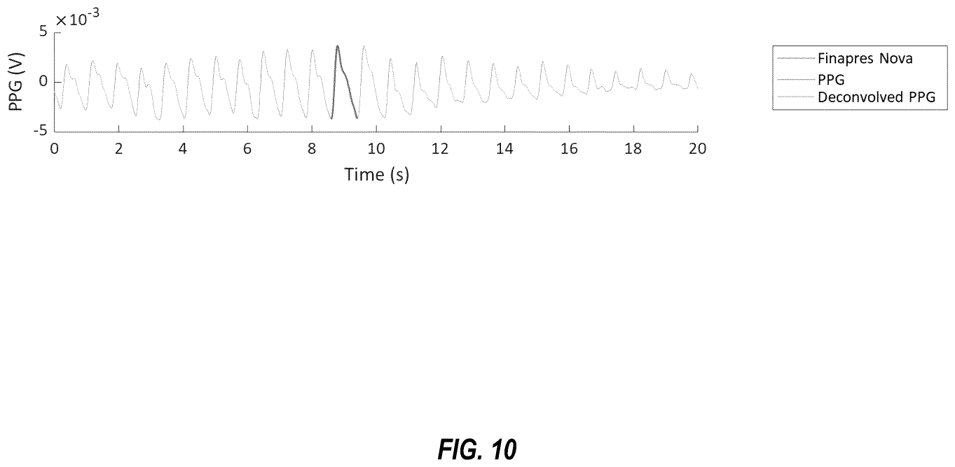

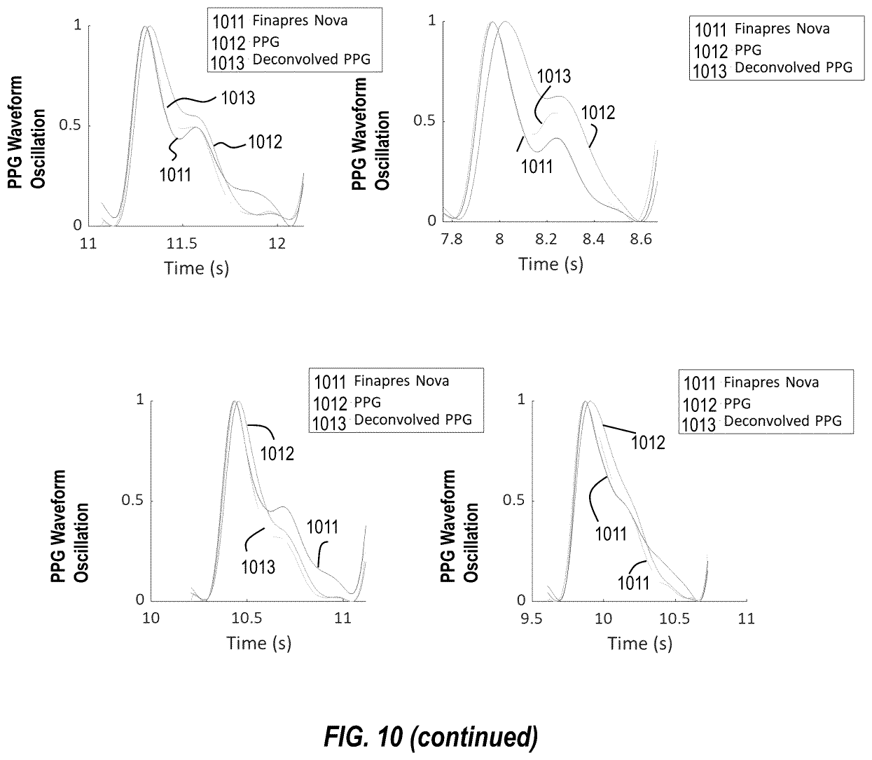

The shape of the BP waveform beat can instead be obtained through deconvolution of the PPG beat at maximum oscillation with the transfer function, as shown in FIG. 10. This beat could be used to reconstruct the entire BP waveform during the finger pressing and/or used in more advanced algorithms to compute BP. For instance, features from the computed BP waveform beat could be used as input to a machine learning model to compute BP. Alternatively, the absolute BP waveform beat can be determined using deconvolution. First, a popular algorithm is applied to compute systolic and diastolic BP. Then, the PPG beat at maximum oscillation is calibrated to these BP levels. Next, the transfer function is deconvolved from the beat. Finally, the maximum and minimum of the deconvolved beat are detected to arrive at systolic and diastolic BP corrected for finger artery viscoelasticity.

Since even larger arteries have some viscous components, the models and methods presented above can also be applicable to refine the BP computation accuracy of automatic cuff devices.

### BP Computation Methods: Employing a Patient-Specific Physiologic Model

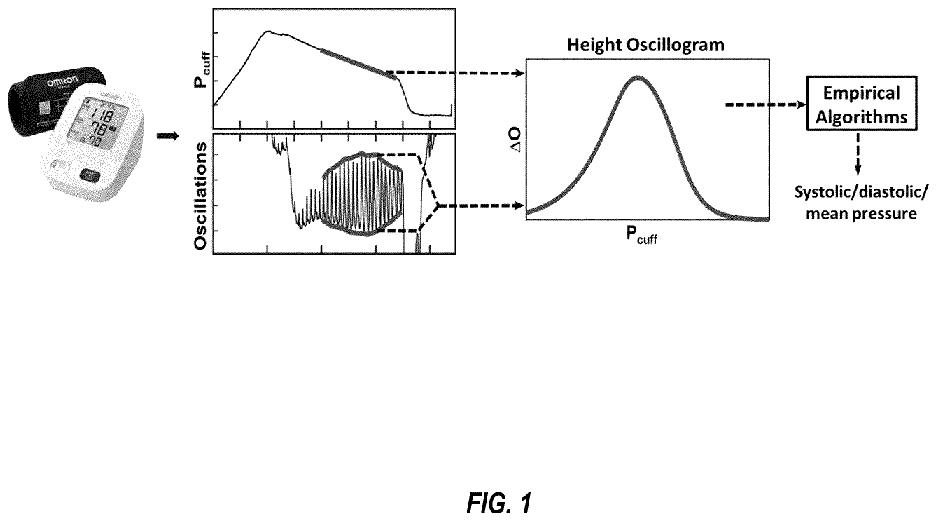

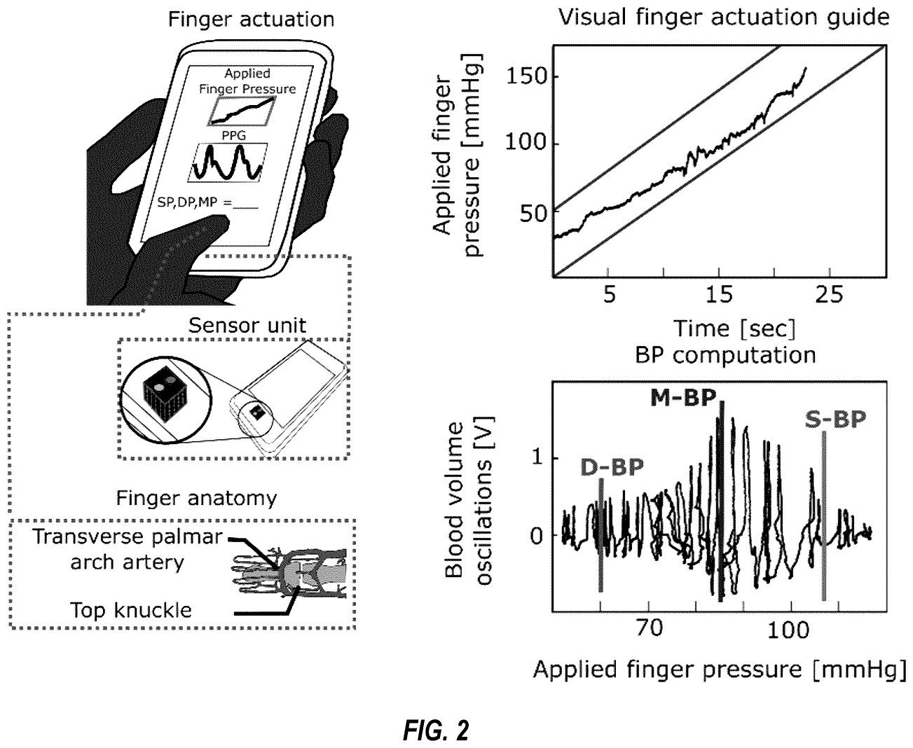

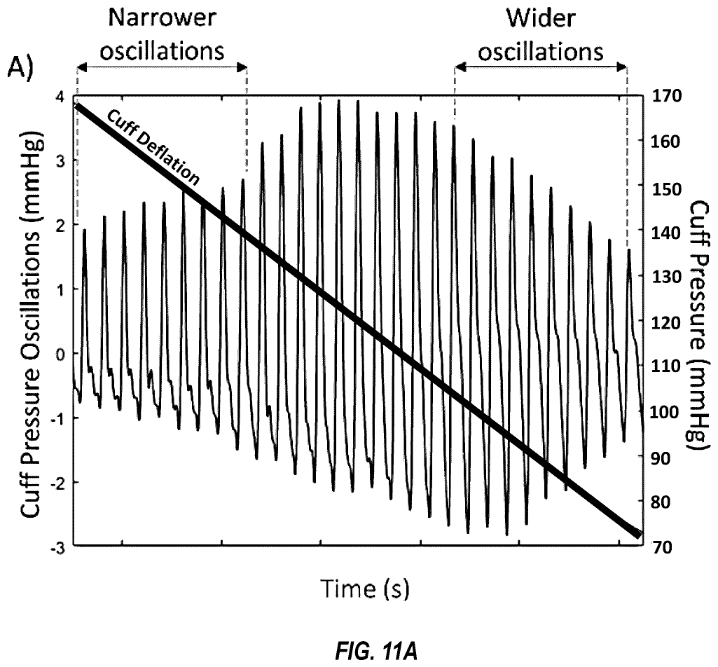

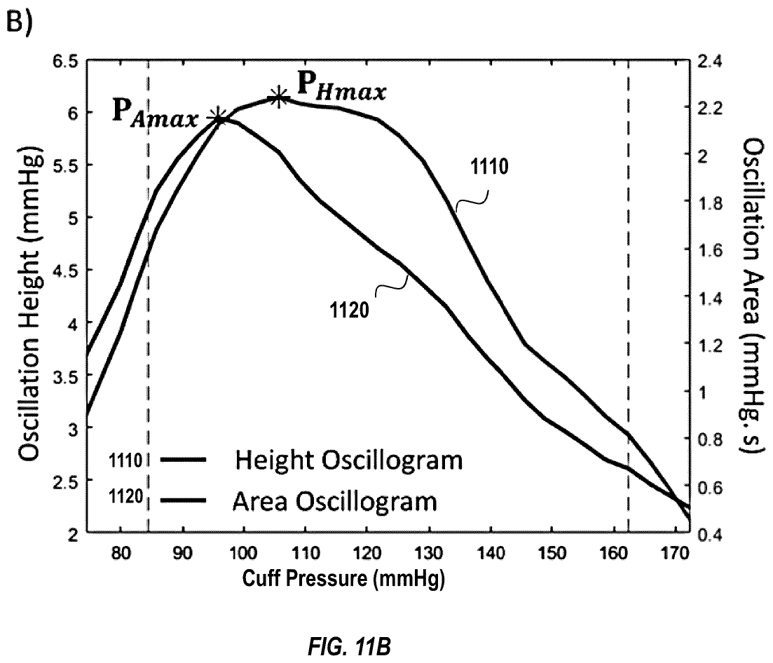

Traditionally, oscillometric devices rely solely on the variations in height (amplitude) of the oscillations as a function of external pressure (i.e., ‘height oscillogram’) for BP computation (see FIG. 1). However, the shape of the oscillations also changes with external pressure, as shown in FIG. 11. The oscillations are narrower at higher external pressures and become wider at lower external pressures (see FIG. 11A). The variation in the area of the oscillations above the diastolic level as a function of external pressure (i.e., ‘area oscillogram’) is left-shifted in comparison to the height oscillogram with a different peak position (see FIG. 11B). The area oscillogram and its peak position can thus offer additional BP information over their height counterparts. The disclosed subject matter employed a physiologic model to develop and validate formulas for the area oscillogram. The patient-specific methods for more accurate BP computation from the oscillometric measurements based on the formulas were conceived. Model-based methods are preferred to machine learning methods when the training data are limited. For instance, there can be limited training data when using gold standard invasive brachial BP as the reference method.

### Model-Based Oscillogram Formulas

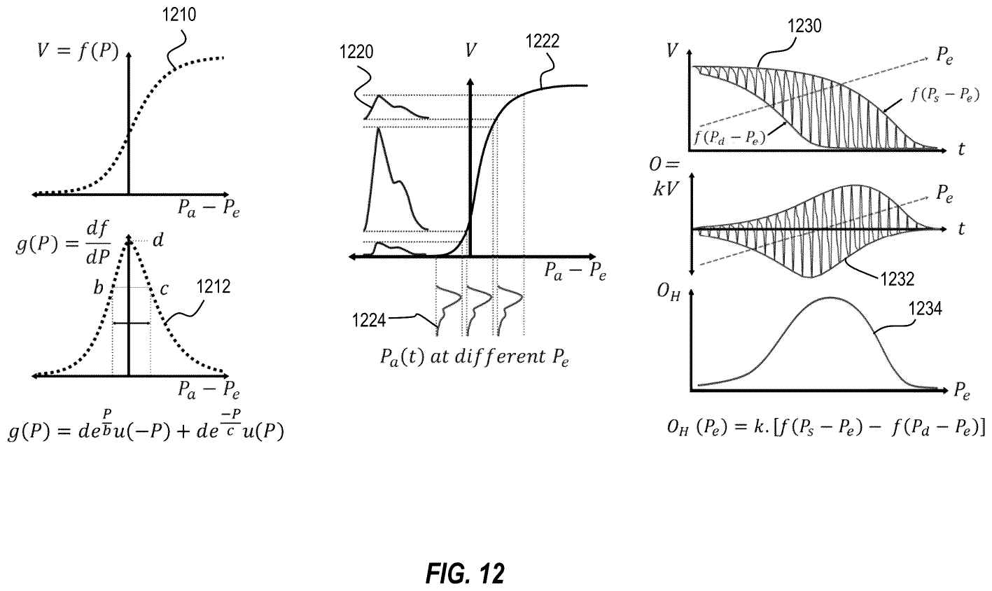

The disclosed subject matter utilizes a physiologic model of oscillometry [5] to establish formulas for the area oscillogram. This model is based on the sigmoidal blood volume-transmural pressure relationship of the artery, as described above and shown again in FIG. 12. The model employs an (asymmetric) exponential function to parameterize the arterial compliance curve (g(P), derivative of sigmoidal relation). This function is defined by the parameters b and c, which denote the widths of the compliance curve in the negative and positive transmural pressure ranges respectively, and the parameter d, which indicates the amplitude of the compliance curve. A similar model was previously used to develop formulas for the height oscillogram [6]. The formula for the height oscillogram (O_{H}(Pₑ)) can be readily obtained from FIG. 12 and is shown in Eq. 1:

$${O}_{H} ( {P}_{e} ) = k d ( c {e}^{- \frac{{P}_{d} - {P}_{e}}{c}} - c {e}^{- \frac{{P}_{s} - {P}_{e}}{c}} ) u ( {P}_{d} - {P}_{e} ) + k d ( ( b + c ) - {be}^{\frac{{P}_{d} - {P}_{e}}{b}} - {ce}^{- \frac{{P}_{s} - {P}_{e}}{c}} ) ( u ( {P}_{e} - {P}_{d} ) - u ( {P}_{e} - {P}_{s} ) ) + k d ( b {e}^{\frac{{P}_{s} - {P}_{e}}{b}} - b {e}^{\frac{{P}_{d} - {P}_{e}}{b}} ) u ( {P}_{e} - {P}_{s} ) \qquad ( Eq . 1 )$$

where k is a scalar to map blood volume oscillations to cuff pressure or PPG oscillations.

Other parametric functions of the arterial compliance curve such as the (asymmetric) exponential-linear function could similarly be used and can afford a more accurate representation [6], but the exponential function was selected here for simplicity.

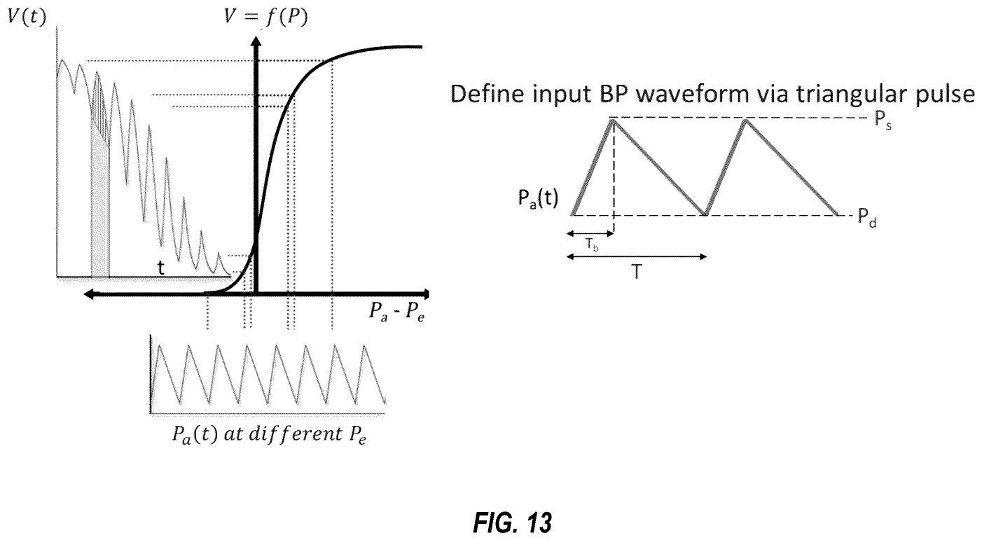

The approach for developing a mathematical model of the area oscillogram is shown in FIG. 13. A triangular waveform was assumed for the shape of the arterial BP pulse (for simplicity). This waveform was inputted into the sigmoidal relation to generate the measured oscillations. The area under each oscillation above the diastolic level can then be computed by straightforward integration.

This leads to a formula for the area oscillogram (O_{A}(Pₑ)) as shown in Eq. 2:

$${O}_{A} ( {P}_{e} ) = k ( \frac{d {c}^{2} T}{{P}_{s} - {P}_{d}} [ {e}^{( \frac{- ( {P}_{s} - {P}_{e} )}{c} )} - {e}^{( \frac{- ( {P}_{d} - {P}_{e} )}{c} )} ] + {dcTe}^{( \frac{- ( {P}_{d} - {P}_{e} )}{c} )} ) u ( {P}_{d} - {P}_{e} ) + k ( ( \frac{d {b}^{2} T}{{P}_{s} - {P}_{d}} [ 1 - {e}^{( \frac{( {P}_{d} - {P}_{e} )}{b} )} ] ) + ( \frac{d {c}^{2} T}{{P}_{s} - {P}_{d}} [ {e}^{( \frac{- ( {P}_{s} - {P}_{e} )}{c} )} - 1 ] + d ( b + c ) T \frac{{P}_{s} - {P}_{e}}{{P}_{s} - {P}_{d}} - {dbTe}^{( \frac{{P}_{d} - {P}_{e}}{b} )} ) ) ( u ( {P}_{e} - {P}_{d} ) - u ( {P}_{e} - {P}_{s} ) ) + k ( \frac{d {b}^{2} T}{{P}_{s} - {P}_{d}} [ {e}^{( \frac{( {P}_{s} - {P}_{e} )}{b} )} - {e}^{( \frac{( {P}_{d} - {P}_{e} )}{c} )} ] - {dbTe}^{( \frac{( {P}_{d} - {P}_{e} )}{b} )} ) u ( {P}_{e} - {P}_{s} ) \qquad ( Eq . 2 )$$

The peak of the area oscillogram is left-shifted in comparison to that of the height oscillogram (see FIG. 11B). The formulas were derived for the peak position by finding the root of the derivative of the oscillogram formulas.

The peak position of the height oscillogram (P_{Hmax}) is shown in Eq. 3:

$$\frac{d ( {O}_{H} )}{d {P}_{e}} = 0 \to {P}_{H max} = {P}_{d} + b [ \frac{P P}{b + c} ] , P P = {P}_{s} - {P}_{d} \qquad ( Eq . 3 )$$

The peak position of the area oscillogram (P_{Amax}) is shown in Eq. 4:

$$\frac{d ( {O}_{A} )}{d {P}_{e}} = 0 \to ( b + P P ) {e}^{( \frac{{P}_{d} - {P}_{A max}}{b} )} + {ce}^{( \frac{{P}_{A max} - {P}_{s}}{c} )} - ( b + c ) = 0 \qquad ( Eq . 4 )$$

Eq. 4 needs to be numerically solved to determine the P_{Amax} value.

Based on nominal parameter values of b and c obtained through a previous study [6], it is assumed that the first term to the right of the arrow is larger than the middle term. As a result, Eq. 4 becomes simplified as shown in Eq. 5:

$${P}_{A max} \approx {P}_{d} + b ln [ \frac{b + P P}{b + c} ] \qquad ( Eq . 5 )$$

Comparing Eqs. 3 and 5, the model predicts that P_{Amax} will be smaller than P_{Hmax} for nominal arterial compliance parameter values.

### Validation of Formulas

De-identified patient data [7], [8] were used to validate the formulas of Eqs. 1-5. The data comprise brachial cuff pressure waveforms from 109 cardiac catheterization patients (58±15 years old, 73% male). Invasive brachial BP measurements were also recorded in the patients. Two oscillometric cuff pressure waveforms were recorded via consecutive inflation-deflation cycles for most patients. A subset of patients (N=39) had the measurements at a baseline condition and after administration of nitroglycerin to reduce BP. Data with artifacts (motion, arrhythmia, etc.) were excluded based on visual inspection. A total of 158 measurements remained for analysis. The cuff pressure waveforms underwent a series of signal processing steps to extract the height oscillogram [6] and area oscillogram (integral of instantaneous pulse height over the pulse duration as a function of cuff pressure).

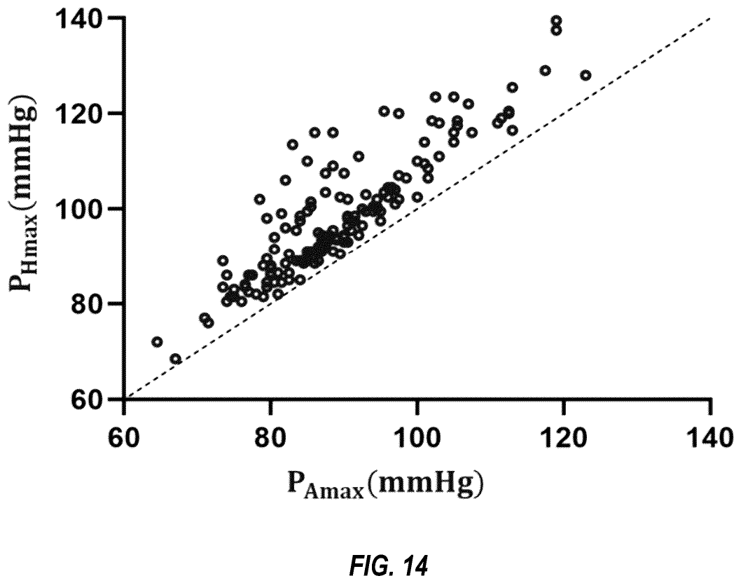

The peak positions of the area and height oscillograms were then detected. FIG. 14 shows that measured P_{Hmax} is always higher than measured P_{Amax}. This experimental result is consistent with the predictions of the formulas in Eqs. 3 and 5.

The area and height oscillogram formulas in Eqs. 1 and 2 inserted with invasive brachial systolic and diastolic BP for Pₛ and P_{d} were then assessed by fitting them to the area and height oscillograms extracted from the 158 waveforms. The predictive value of each formula was evaluated in terms of the normalized RMS error of the model fits.

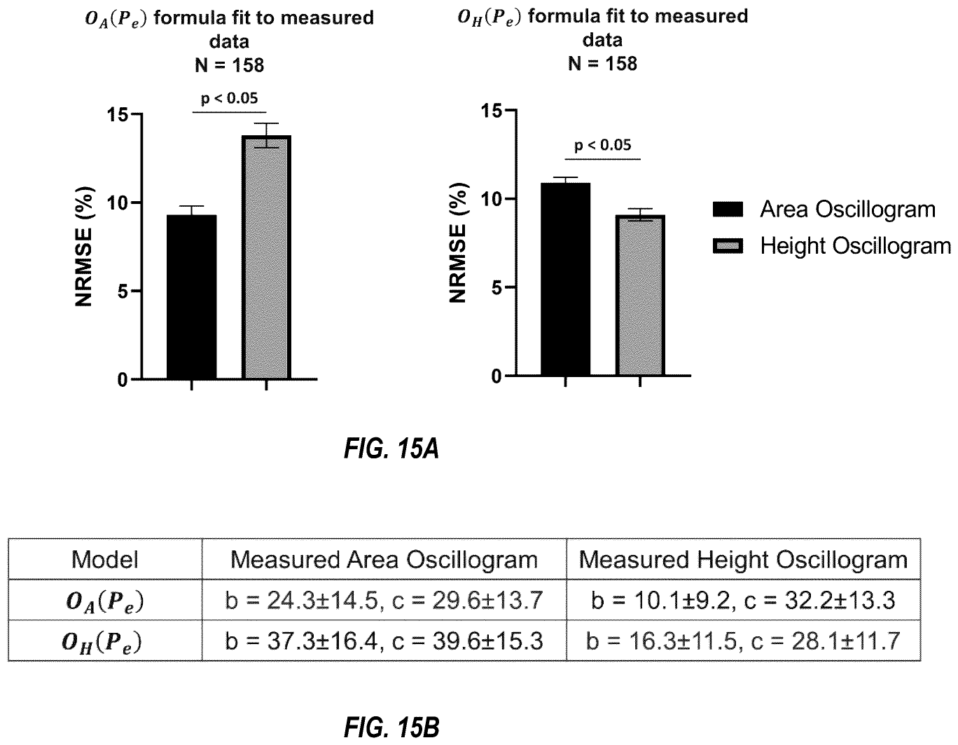

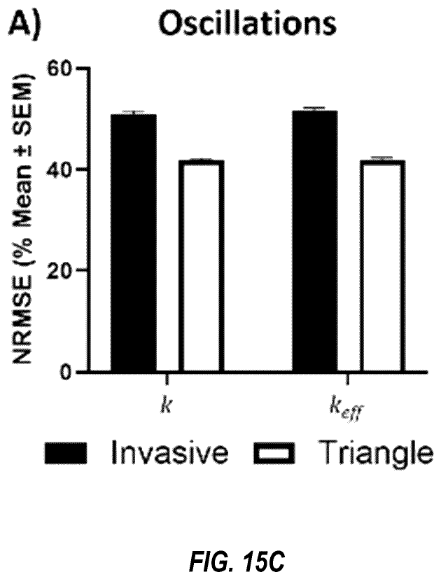

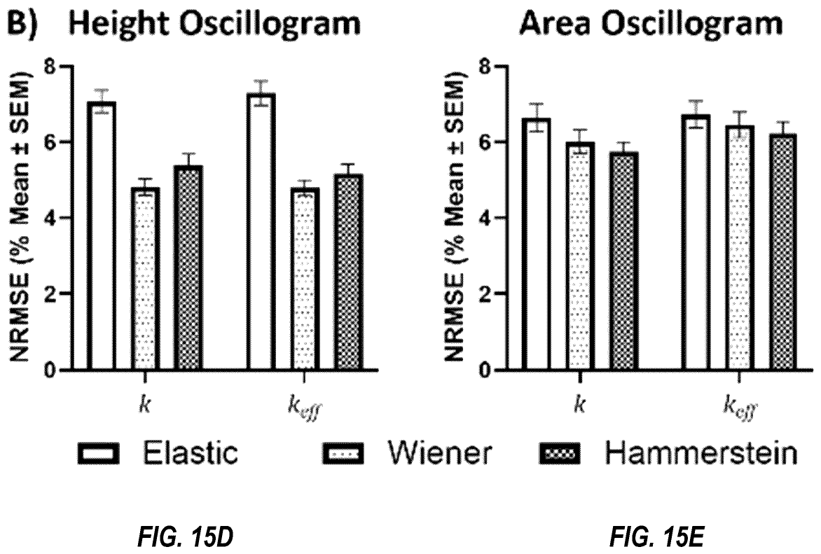

FIG. 15A shows that the O_{A}(Pₑ) formula fit the area oscillograms significantly better than the height oscillograms, while the O_{H}(Pₑ) formula fit the height oscillograms significantly better than the area oscillograms. FIG. 15B shows the resulting b and c parameter estimates. The b and c parameter values obtained from fitting the O_{A}(Pₑ) formula to the area oscillograms and the O_{H}(Pₑ) formula to the height oscillograms are more consistent with the literature [6]. These results help validate the oscillogram area formula.

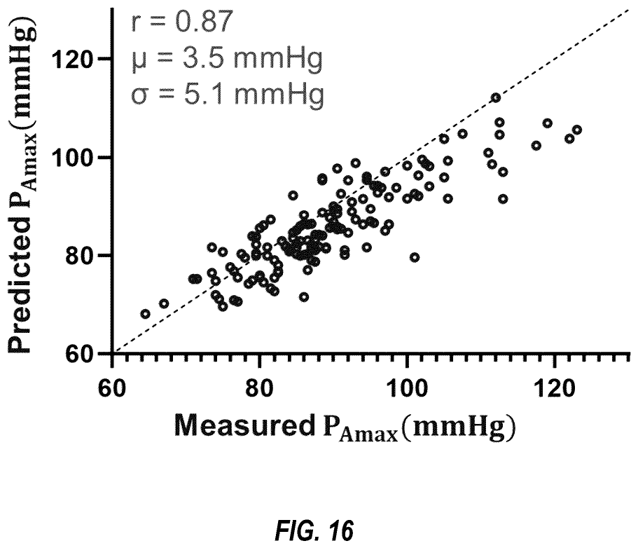

Finally, Eq. 4 was inserted with invasive brachial systolic and diastolic BP for Pₛ and P_{d} and b and c values obtained from fitting the O_{H}(Pₑ) formula to the height oscillograms. The equation was then numerically solved to obtain P_{Amax} for each measurement. FIG. 16 shows the predicted P_{Amax} correlated well with the corresponding measured peak position of the area oscillogram. These results help validate the formula for the peak position of the oscillogram area.

In sum, the formulas of Eqs. 1-5 predict patient data well, and the area oscillogram differs from the height oscillogram.

### Patient-Specific Oscillometric BP Computation

In previous work [5], [9], a height oscillogram formula similar to Eq. 1 was fitted to height oscillograms to estimate the arterial compliance curve model parameters as well as systolic and diastolic BP. Since both BP and arterial stiffness are effectively measured, the method can be specific to the patient at the time of measurement for a ‘patient-specific’ oscillometric BP computation method. However, the uniqueness of the five parameter estimates is a concern. In other words, there cannot be enough information in the height oscillogram to estimate all five parameters.

The main idea is to leverage height, area, and even other shape oscillograms to increase the available information in the measurements without increasing the number of parameters for estimation. For instance, a superior patient-specific method involves optimal fitting of both the area and height oscillogram formulas of Eqs. 1 and 2 to their respective measurements as shown in Eq. 6:

$${min}_{( b , c , kd , {P}_{s} , {P}_{d} )} {\int }_{{P}_{{e}_{min}}}^{{P}_{{e}_{max}}} [ {( {O}_{H} ( {P}_{e} ) - {\int }_{{P}_{d} - {P}_{e}}^{{P}_{s} - {P}_{e}} kd \cdot g ( P , b , c ) dP )}^{2} / {{O}_{H} ( {P}_{e} )}^{2} + {( {O}_{A} ( {P}_{e} ) - [ {\int }_{0}^{T} kd \cdot f ( {P}_{a} ( t ) - {P}_{e} ; b , c ) dt - T \cdot kd \cdot f ( {P}_{d} - {P}_{e} ; b , c ) ] )}^{2} / {O}_{A} {( {P}_{e} )}^{2} ] {dP}_{e} \qquad ( Eq . 6 )$$

In solving this optimization problem, the three parameters b, c, and kd as well as systolic and diastolic BP can be constrained to physiologic levels [5], [9]. The oscillograms can alternatively be normalized to unity amplitude to eliminate the kd parameter and thereby yield a four parameter estimation problem. The model can assume that the cuff transducer relating blood volume oscillations to cuff pressure oscillations is linear. However, the cuff transducer can be more nonlinear in the lower cuff pressure range. The optimization problem can therefore be solved using the measurements only over higher cuff pressure (Pₑ) ranges. For instance, the cuff pressure range can start when the area oscillogram rises to 0.75 of its peak amplitude and end when the height oscillogram falls to 0.5 of its peak amplitude.

Similarly, the P_{Amax} and P_{Hmax} formulas can be used to facilitate patient-specific BP computation. For instance, the two peak position formulas provide constraints that reduce the parameters by two. Moreover and importantly, the peak positions can be robustly measured. For another example, these formulas (see Eqs. 3 and 4) include only four parameters (Pₛ, P_{d}, b, c). Two or more similar formulas pertaining to the variations in other shape features of the oscillations as a function of the external pressure (‘other shape oscillograms’) would yield a system of equations comprising robust measurements that could be solved uniquely to compute systolic and diastolic BP as well as the arterial compliance curve widths.

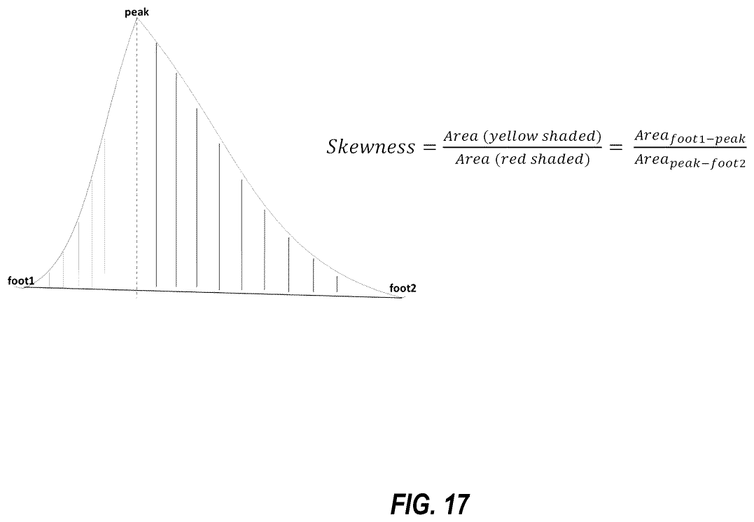

Other oscillation shapes for which formulas can be readily derived include skewness (ratio of oscillation area from foot to peak to oscillation area from peak to foot), as shown in FIG. 17.

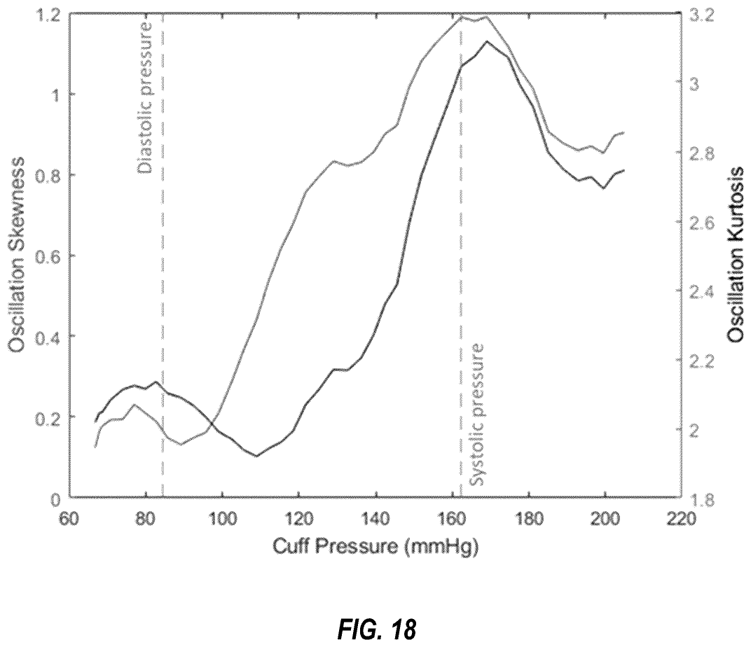

Kurtosis is another oscillation shape that would provide additional information about BP. FIG. 18 shows measured skewness and kurtosis oscillograms. Formulas can be derived to represent the points of slope change.

As mentioned earlier, an (asymmetric) exponential-linear function can better represent the arterial compliance curve [6] and is shown in Eq. 7:

$$g ( P ) = d {e}^{\frac{P}{b}} ( - \frac{P}{b} + 1 ) u ( - P ) + d {e}^{\frac{- P}{c}} ( \frac{P}{c} + 1 ) u ( P ) \qquad ( Eq . 7 )$$

Hence, formulas based on this function can improve accuracy and can be derived readily but are more involved.

The formulas presented here assume purely elastic arteries via the sigmoidal relation. They can thus be best to improve the accuracy of the automatic arm cuff device. However, they could also be applied to facilitate BP computation via the oscillometric finger pressing method. For instance, BP computations resulting from the formulas could be corrected for viscoelasticity as described in any of the ways above. A simultaneous ECG waveform during the finger pressing could be used to help define the beats and thereby construct more accurate area and other shape oscillograms for modeling fitting as well.

### Venous Pressure Measurement Methods

The oscillometric arm cuff principle can also be used to measure venous pressure [10]. A peak in the oscillation amplitude versus cuff pressure function (oscillogram) is sometimes apparent near the venous pressure. However, venous pressure is typically low (e.g., 5-15 mmHg). Therefore, to detect this peak, low cuff pressure needs to be applied. But, low cuff pressures result in poor cuff to arm contact and thus poor signal quality (i.e., no visible oscillations). The oscillogram is thus often incomplete at low cuff pressures and ‘venous oscillometry’ is typically ineffective. The main idea is to make the oscillometric measurement at a location substantially lower than heart level (e.g., the finger or wrist with arms fully lowered) to increase the local pressure via the weight of blood and thus the signal-to-noise ratio and thereby effectively measure venous pressure.

### Concepts and Measurements

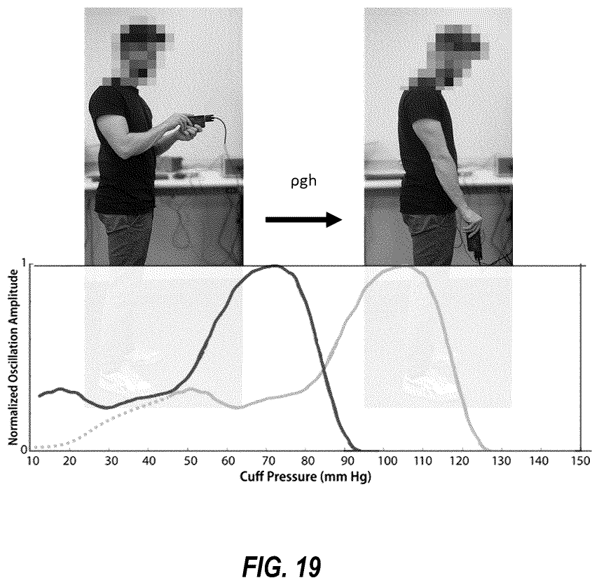

The disclosed subject matter extended the oscillometric finger pressing method to measure both BP and venous pressure. A smartphone including a PPG-force sensor unit is used. The force sensing contact area needs to be small (e.g., 5 mm diameter circle) to accurately measure low finger pressure. The smartphone is held with hands fully lowered to increase finger BP by ρgh (where ρ is the known blood density, g is gravity, and h is approximately the arm length), and finger pressing is performed. FIG. 19 illustrates that the oscillogram is shifted to the right by ρgh. The contact pressure of the sensor on the skin can determine the PPG noise level, whereas the transmural pressure determines the PPG signal level. Thus, by shifting the oscillogram by ρgh, it is possible to measure an oscillogram with superior PPG signal-to-noise ratio at low finger pressures. Using this principle, a maximum PPG oscillation amplitude is usually visible at a finger pressure around the venous pressure (approx. 5-15 mmHg for healthy individual) plus ρgh. The venous pressure (and even BP) is then obtained by subtracting ρgh from the detected finger pressure. Note that arm length could be approximated by the user height for greater convenience.

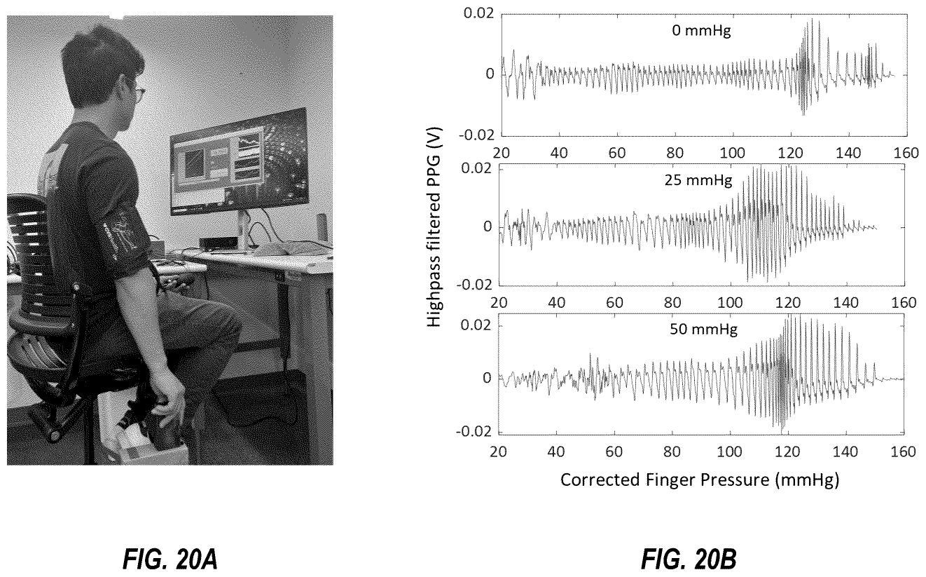

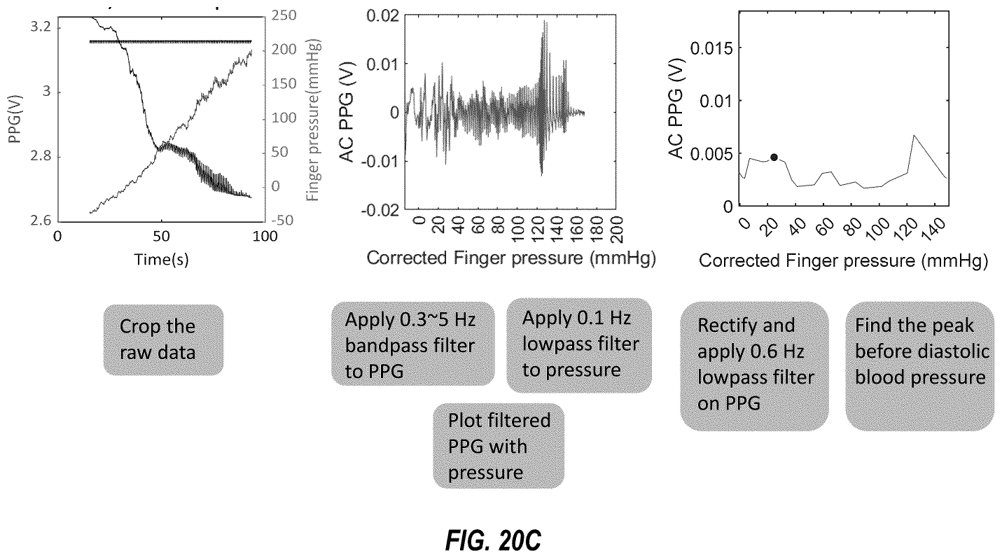

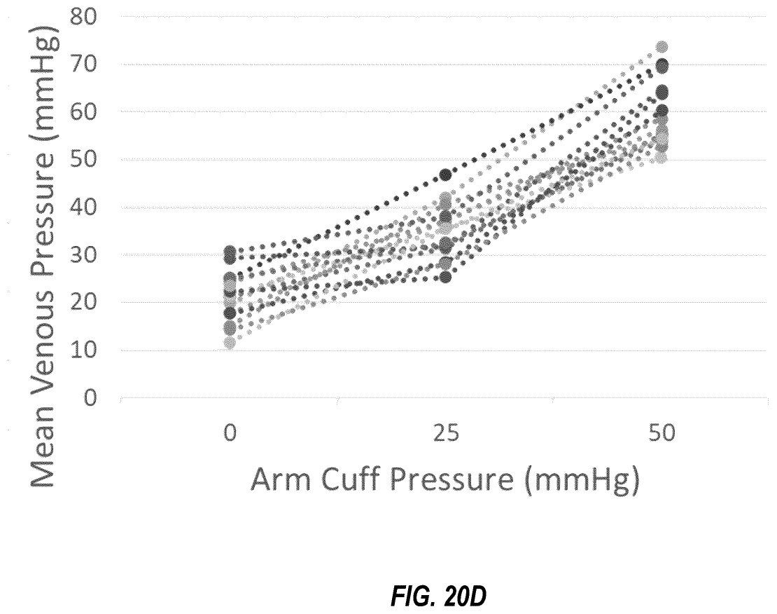

To test the oscillometric finger pressing method for measuring venous pressure, a benchtop system was employed. The system included a handheld device to measure finger PPG and finger pressure and a monitor to visually guide the finger pressing. Fifteen volunteers obtained measurements with the device. As shown in FIG. 20A, each participant performed the finger pressing with their hand fully lowered while wearing a manual arm cuff on the same limb. The participants performed the finger pressing with the arm cuff inflated to 0, 25, and 50 mmHg to increase the venous pressure in the finger via venous pooling. More specifically, finger venous pressure will be at least 25/50 mmHg when the arm cuff pressure is set to 25/50 mmHg. FIG. 20B shows the bandpass filtered PPG signal as a function of corrected finger pressure (i.e., finger pressure minus measured ρgh) from one participant. The maximum low-pressure variations, which correspond to mean venous pressure, is at about 25, 30, and 55 mmHg for arm cuff pressure at 0, 25, and 50 mmHg. The low-pressure variations are caused by both venous oscillations and the sharp DC PPG change due to venous collapse (see next paragraph). FIG. 20C shows an exemplary automated algorithm to detect the low-pressure variations. The algorithm plots the bandpass filtered PPG signal against the corrected finger pressure and then applies rectification followed by lowpass filtering to detect the maximum variation at low pressure. FIG. 20D shows the mean venous pressure detected with this algorithm for each participant at each arm cuff pressure. The method was able to consistently track the venous pressure.

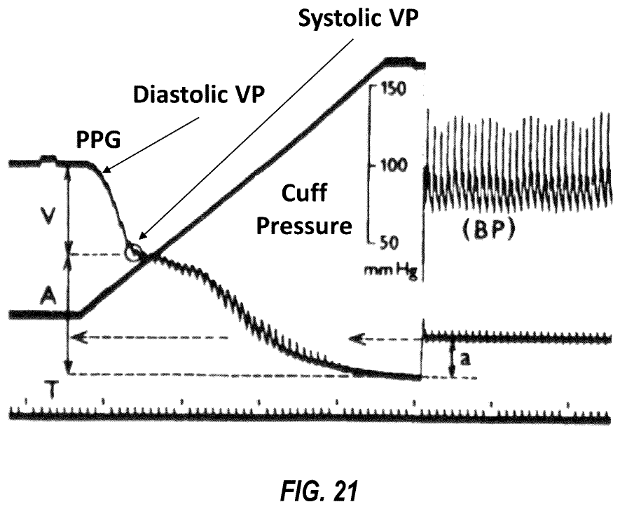

FIG. 21 shows that only the DC PPG signal as a function of finger pressure could instead be used to detect venous pressure. The plot shows an initial steep drop in PPG amplitude due to the small venous pulse pressure followed by the common pattern of arterial oscillometry. The two fiducial points on the steep drop can indicate diastolic and systolic venous pressure (VP).

The method for measuring venous pressure can be extended to any type of device that measures BP at a distal location. A wrist automatic cuff device, a finger automatic cuff-PPG device, or a finger worn ring with PPG-force sensor unit are example devices.

### Example 1: Nonlinear Viscoelastic Modeling of Finger Arteries

Oscillometric finger pressing is a smartphone-based blood pressure (BP) monitoring method. Finger photoplethysmography (PPG) oscillations and pressure are measured during a steady increase in finger pressure, and an algorithm computes systolic BP (SP) and diastolic BP (DP) from the measurements. The objective was to assess the impact of finger artery viscoelasticity on the BP computation.

Here, nonlinear viscoelastic modeling was conducted in the context of the oscillometric finger pressing method. The extent of viscoelasticity was quantified by fitting viscoelastic and purely elastic models to finger oscillometric measurements. As finger artery viscoelasticity is also important, a model parametric sensitivity analysis was performed to reveal the adverse impact of viscoelasticity on a popular elastic model-based oscillometric BP computation algorithm. Finally, the disclosed subject matter provides a potential remedy toward accurately computing BP despite the finger viscoelastic effect.

### Data Collection for Model Building

Physiologic data were collected from 15 healthy participants. The participant demographics were as follows: 33% female, 31±12 (mean±standard deviation) years of age, 75±11 kg in weight, and 172±8 cm in height. A custom benchtop system, consisting of an infrared reflectance-mode PPG sensor on top of a load cell was utilized to measure finger arterial volume oscillations and applied pressure, as shown in FIG. 3. The PPG and applied pressure data were collected at 1000 Hz using a commercial data acquisition unit (USB-6003, NI). The participants were guided with visual feedback on a monitor to linearly increase finger pressure over time. Each participant performed the finger pressing method while holding the sensor-unit at heart level. A finger arterial BP waveform was recorded simultaneously via a finger cuff volume-clamp device (NOVA, Finapres) at 200 Hz, as also shown in FIG. 3. Electrodes in lead I configuration were used to record an ECG waveform via the NOVA and the NI analog inputs for synchronization purposes. The collected data were down sampled to 100 Hz for further analysis.

### Model Input and Output

The finger artery system for identification relates the transmural pressure (P_{T}(t)) to the blood volume in the artery (V(t))·(P_{T}(t)) of an artery, defined as the internal BP (Pₐ(t)) minus the external pressure (Pₑ(t)), is measured in the experimental setup (FIG. 3). While absolute blood volume cannot be measured, PPG can measure a waveform proportional to the arterial blood volume oscillations (PPG_{ac}(t)). The PPG_{ac} waveform is therefore a highpass filtered version of V(t) multiplied by an unknown constant (k). The identifiable system thus comprises the nonlinear dynamics of the artery in cascade with the highpass filter and PPG scaling, as shown in FIG. 4, where P_{T}(t) is the input and PPG_{ac}(t) is the output of the system. The highpass filter is known and the same as the one used for filtering the raw PPG measurements (first order Butterworth filter with cutoff frequency of 0.3 Hz). Only the nonlinear dynamic model (as well as the PPG scaling) is identified from P_{T}(t) and PPG_{ac}(t).

### Viscoelastic and Elastic Models

A Hammerstein model (static nonlinearity followed by linear dynamics) and a Wiener model (linear dynamics followed by static nonlinearity) were employed to represent arterial nonlinear viscoelasticity. These models are typical representations of nonlinear dynamics that are often encountered in biological systems and are easily identifiable. Although they can be part of the black-box model family, they were quite interpretable models here, since they were designed with knowledge of arterial physiology. In particular, the static nonlinearity in both models is a sigmoidal function, which is known to enable good fitting of oscillometric arm cuff measurements and has parameters that carry physiological meaning. The impact of the difference in the structural order of the static nonlinearity and linear dynamics depends on their specific characteristics. However, in general, the Wiener representation needs to be preferred when the system dynamics vary with the operating point. On the other hand, when only the system gain varies with the operating point, the Hammerstein model generally outperforms the Wiener representation.

The sigmoidal function ƒ(P_{T}) is specifically given as follows:

$$df ( {P}_{T} ) / {dP}_{T} = g ( {P}_{T} ) = {ae}^{( \frac{{P}_{T}}{b} )} ( - \frac{{P}_{T}}{b} + 1 ) u ( - {P}_{T} ) + {ae}^{( \frac{{P}_{T}}{b} )} ( \frac{{P}_{T}}{b} + 1 ) u ( {P}_{T} ) \qquad ( Eq . 8 )$$

$$f ( {P}_{T} ) = {ae}^{( \frac{{P}_{T}}{b} )} ( 2 b - {P}_{T} ) u ( - {P}_{T} ) - [ {ae}^{( - \frac{{P}_{T}}{b} )} ( 2 c - {P}_{T} ) u ( - {P}_{T} ) + 2 a ( b + c ) ] u ( {P}_{T} ) \qquad ( Eq . 9 )$$

Here, g(P_{T}) is the unimodal arterial compliance curve and is defined by an exponential-linear function with u(·) representing the unit step function, parameter a indicating the function height at zero transmural pressure, and parameters b and c denoting the function widths over the negative and positive transmural pressure regimes. For simplicity, the linear dynamics are represented with a unity gain single pole filter H(s). The filter has one parameter (d) denoting its pole location. FIGS. 5A-5C show the Hammerstein and Wiener viscoelastic models.

For comparison, a purely Elastic model was also employed. This model consists of just ƒ(P_{T}), as also shown in FIGS. 5A-5C.

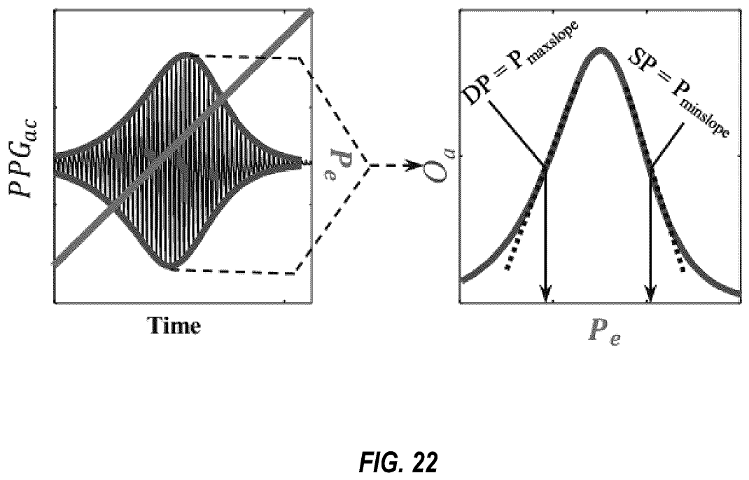

Note that the unknown k scale factor for mapping the unobserved V(t) to the measured PPG_{ac}(t) (FIG. 22) can be absorbed by the a parameter. So, the model parameters for estimation are e=a·k, b, and c for all three models along with d for the Hammerstein and Wiener models.

### Model Fitting

The parameters were estimated for each model and participant by fitting the estimated PPG_{ac} output of the model driven by the measured transmural pressure input to the measured PPG_{ac} output in the least squares sense. A trust-region optimization method, implemented with the Matlab fmincon function, was specifically applied to estimate e and d or e alone for each pair of b and c between 1 and 20 mmHg in unity increments. These parameter ranges encompass the typical parameter values for the brachial artery, which are expected to be larger than the parameter values for thin finger arteries. Then, the best fitting model was selected amongst the candidates. Although classic system identification tools could have been leveraged to estimate the d parameter for the Hammerstein model, such tools were not applicable to the Wiener model due to the unobservable V(t). Therefore, the same estimation method was used for all models for a fair comparison.

Since volume-clamp devices can yield appreciable error in the mean value of the finger BP waveform, an offset to the mean value of the waveform was included as another parameter for estimation. This parameter was estimated through grid search from −10 to 10 mmHg in unity increments. This additional free parameter was required for satisfactory model fitting to the oscillograms.

### Model Evaluation

Each model for each participant was evaluated in terms of the normalized root-mean-square-error of the PPG_{ac} fit (RMSE) and the normalized root-mean-square-error of the oscillogram fit (RMSE_{O}). The normalizing term is the root-mean-square of the measurement (PPG_{ac} or oscillogram) so that both errors have percent units. The measured oscillograms were computed as the peak-to-peak amplitude of the PPG_{ac} waveform followed by five-beat median filtering to mitigate measurement and respiratory artifacts. The errors for each model were statistically compared using the Wilcoxon signed rank test.

### Model Simulations

To understand the impact of finger artery viscoelasticity on the BP computation, a model parametric sensitivity analysis was performed. The median of the parameter estimates of each participant and model was used for simulation. A simulated finger BP waveform Pₐ(t) and a 60-second linear increase from 10 to 200 mmHg in finger pressure Pₑ(t) were used to form the P_{T}(t) input. Pₐ(t) was constructed via a truncated Fourier series model as follows:

$${P}_{a} = DP + 0.5 PP + 0.36 PP ( sin ( ω t ) + \frac{1}{2} sin 2 ( ω t ) + \frac{1}{4} sin 3 ( ω t ) ) \qquad ( Eq . 10 )$$

where ω is the angular frequency of heart rate, which was fixed at 60 beats per minute.

The output of the model, the simulated blood volume V(t), was then highpass filtered. The oscillogram was constructed by plotting the simulated oscillation amplitudes (Oa) versus the simulated Pₑ. The finger pressures at minimum and maximum slopes of the simulated oscillogram (Pₘᵢₙₛₗₒₚₑ, Pₘₐₓₛₗₒₚₑ) were used to estimate SP and DP, respectively, as shown in FIG. 22. This derivative algorithm is popular in oscillometry and can be particularly effective for finger arteries according to the elastic model.

A number of simulations was performed to compute the sensitivity of the BP computation errors of the derivative algorithm to up to +50% variations in the PP and b, c or d parameters.

### Model Comparisons—Representative Participant

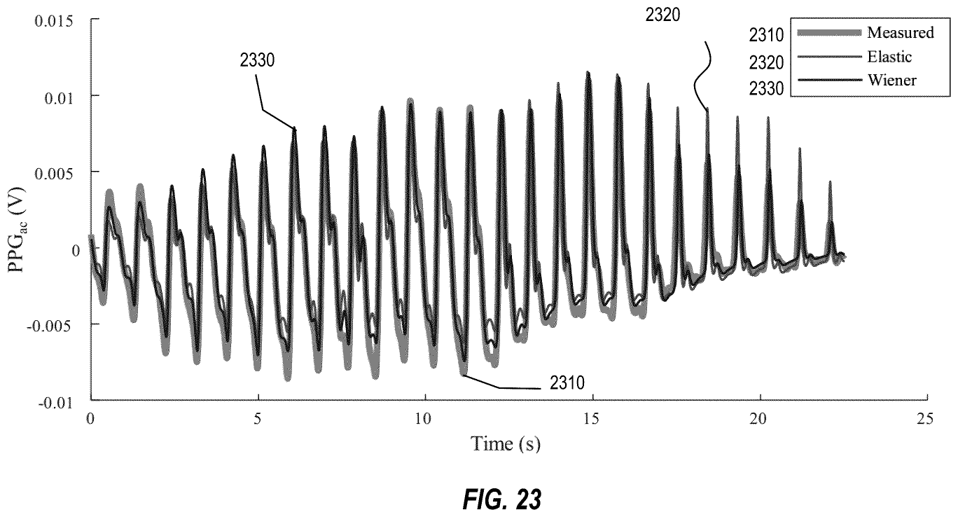

Representative sample (median fitting error result) of estimated PPG_{ac} waveforms via the Wiener and the Elastic models are benchmarked against the measured PPG_{ac} waveform from a participant in FIG. 23. The plot demonstrates better agreement between the estimated and measured waveforms for the viscoelastic model (similar results were obtained with the Hammerstein model but not included for clarity). The following general observations can be made from the sample: (1) the viscoelastic model results in a smoother PPG_{ac} waveform (similar to the measured waveform) with more damped second and third waveform peaks, and (2) the PPG_{ac} amplitude at high finger pressure (toward the end of the waveform) is better represented by the viscoelastic model. For this participant, the RMSE values were 18.8%, 21.7%, and 46.9% for the Hammerstein, Wiener, and Elastic models, respectively.

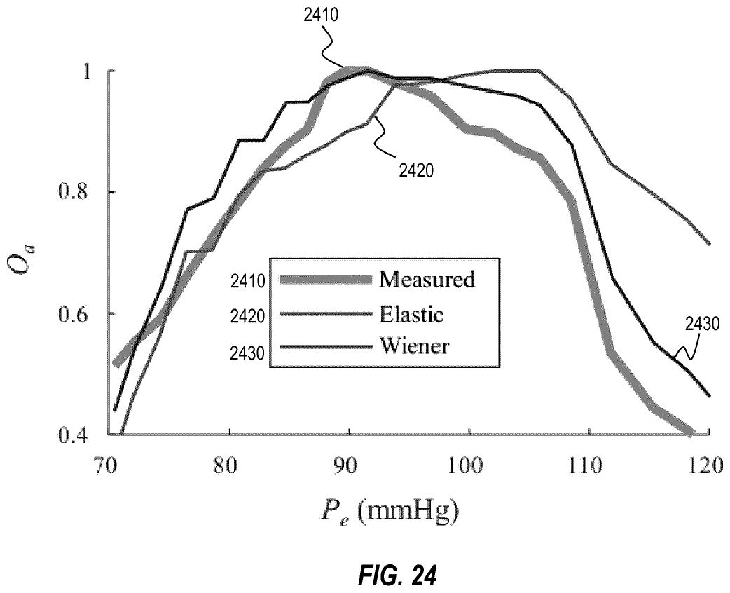

The corresponding estimated and measured oscillograms are shown in FIG. 24. This plot shows that the Elastic model is not able to fit the oscillogram well to the right of the maximum oscillation amplitude, whereas the viscoelastic model captures the oscillation amplitude better. For this participant, the RMSE_{O} values were 7.2%, 5.9%, and 13.6% for the Hammerstein, Wiener, and Elastic models, respectively.

### Model Comparisons All Participants

The model fitting errors of the estimated versus measured PPG_{ac} waveform computed across the 15 participants are shown in FIG. 6A. The plot includes data points representing the RMSE values for each participant. The RMSE values (median (interquartile range)) were 29.8% (26.9%-33.8%), 30.5% (25.6%-34.0%), and 50.9% (46.7%-53.7%) for the Hammerstein, Wiener, and Elastic models, respectively. The RMSE values of both viscoelastic models were significantly lower than the RMSE value of the Elastic model (p<0.01). More importantly, the error was reduced by almost half, which means the one additional parameter in the viscoelastic models (d) was important in modeling the arterial transmural pressure-volume relationship and did not merely help in fitting noise.

The corresponding model fitting errors of the estimated versus measured oscillograms are shown in FIG. 6B. The [symbol] RMSE [symbol] _O values were 6.4% (5.0%-8.2%), 5.9% (4.5%-7.9%), and 13.6% (9.1%-17.5%) for the Hammerstein, Wiener, and Elastic model-based estimates of systolic BP (SP) and diastolic BP (DP), respectively. The RMSE_{O} values were significantly lower for both viscoelastic models (p<0.01), and the error reduction was likewise about a half. While the RMSE_{O} values for the two viscoelastic models were comparable, the RMSE_{O} value for the Wiener model was statistically lower than the RMSE_{O} value for the Hammerstein model (p<0.05).

The estimated b and c model parameters for all participants are shown in FIGS. 6C and 6D. The b and c parameters were not significantly different between the models and were about 8 and 12 mmHg, respectively.

The estimated d model parameter of the viscoelastic models for all participants is shown in terms of transfer function cutoff frequency in FIG. 6E. For both models, the cutoff frequency was about 3 Hz, indicating a significant damping effect.

The viscoelastic models thus fit the measurements much better than the Elastic model. However, while the Wiener model did yield lower oscillogram fitting error than the Hammerstein model, the two models produced similar results overall.

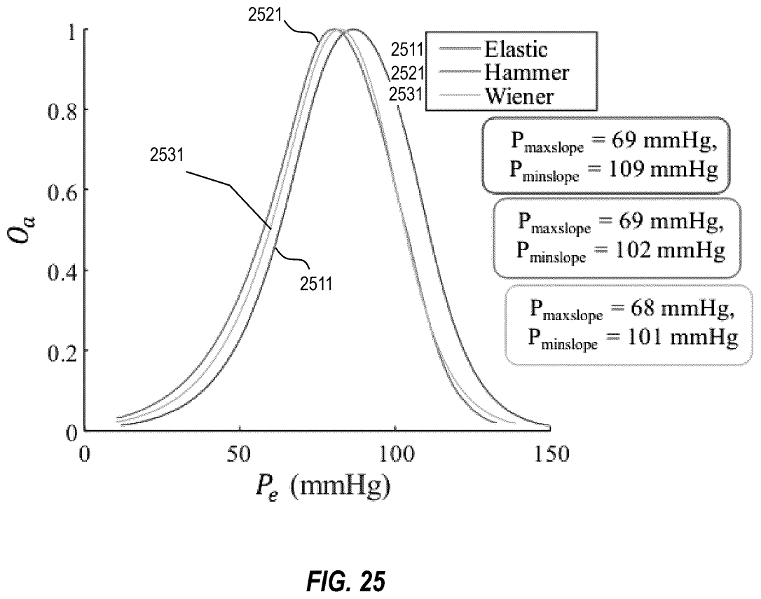

Toward a further comparison to distinguish between the viscoelastic models, FIG. 25 shows the simulated oscillograms from all three models with median parameter values when driven with a simulated transmural pressure waveform. The oscillograms via the viscoelastic models were compressed compared to the Elastic model. If the Hammerstein model with linear dynamics following the static nonlinearity were better, first deconvolving the estimated linear transfer function from the PPG_{ac} waveform and then constructing the oscillogram would yield an oscillogram that is expanded much like the simulated oscillogram via the Elastic model. On the other hand, if the Wiener model were better, such deconvolution can have less impact on the oscillogram due to the linear dynamics preceding the static nonlinearity.

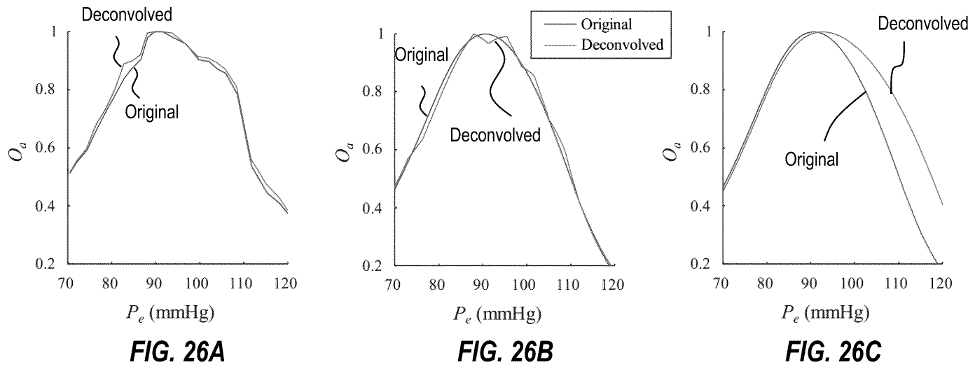

FIG. 26A shows experimental oscillograms from a representative participant. The plot specifically displays the constructed oscillogram from the original measured PPG_{ac} waveform and the oscillogram after deconvolving the waveform with the estimated transfer function. The deconvolution had little impact on the oscillogram. For comparison, FIGS. 26B and 26C show the analogous results for simulated PPG_{ac} waveforms with the Wiener and Hammerstein models, respectively. The participant's estimated model parameters (a, b, c and d) and measured BP and heart rate were used for the simulations. Very similar results are observed for the experimental data and the Wiener model simulation; the oscillogram remains similar after deconvolution. On the other hand, as just explained, deconvolution stretched the oscillogram simulated with the Hammerstein model (i.e., the deconvolution canceled out the effect of damping).

In sum, the Wiener model explains the oscillometric finger pressing data better than the Hammerstein model and much better than the Elastic model.

### Wiener Model Parametric Sensitivity Analysis

The model simulations with median parameter values indicate that viscoelasticity causes the popular derivative algorithm to underestimate SP via Pₘᵢₙₛₗₒₚ but barely affects this algorithm in computing DP via Pmaxslope (FIG. 25).

A parametric sensitivity analysis of the BP estimation errors of the derivative algorithm was performed via further simulations with the Wiener model. FIG. 7A shows the SP estimation errors as a function of PP for different transfer function cutoff frequencies. The underestimation of SP via Pₘᵢₙₛₗₒₚₑ increases with increasing PP and decreasing cutoff frequency (i.e., greater viscoelasticity). FIG. 7B shows that the b parameter only slightly affects the SP estimation error. FIGS. 7C and 7D show the DP estimation errors via Pₘₐₓₛₗₒₚₑ as a function of PP for different transfer function cutoff frequencies and for different c parameters, respectively. These parameters affect the DP computation relatively little.

In sum, finger artery viscoelasticity causes the derivative algorithm to underestimate SP. The SP underestimation is substantial when both PP and the extent of viscoelasticity are large.

### Potential Estimation of the Transfer Function Cutoff Frequency to Compensate for Finger Artery Viscoelasticity

Quantifying the extent of finger artery viscoelasticity from existing measurements could help compensate for its adverse effect on the BP computation. The mean of the normalized PPG_{ac} waveform at the maximum oscillation beat (PPGmean) is a good candidate, as the sigmoidal function can be most linear at zero transmural pressure where the maximum oscillation typically occurs. Therefore, the relationship between arterial volume and pressure can be close to the transfer function alone. Assuming relatively little variability in the finger BP waveform between individuals, PPGₘₑₐₙ can be large when the viscoelastic effect is large (largely damped; “bulky PPG oscillation”) and small when there is little viscoelasticity (purely elastic; “spiky PPG oscillation”).

The oscillometric finger pressing method is a potential method for calibration-free cuffless BP monitoring via widely available smartphones. While the oscillometric principle is proven for brachial artery BP measurement, small finger arteries are more viscoelastic, which could make computing BP from finger oscillometric measurements more challenging.

### Importance of Finger Artery Viscoelasticity

Nonlinear viscoelastic modeling was conducted in the context of the oscillometric finger pressing method. First, viscoelastic and purely elastic models were compared in terms of fitting experimental data. The output of each model was specifically fitted to a measured transmural pressure input to the measured PPG_{ac} waveform during finger pressing from 15 participants. Hammerstein and Wiener viscoelastic models could fit the data with half the error of the Elastic model (FIGS. 6A and 6B). These system identification results indicate that finger artery viscoelasticity needs to be accounted for in the oscillometric finger pressing method.

### Wiener Model of Viscoelasticity

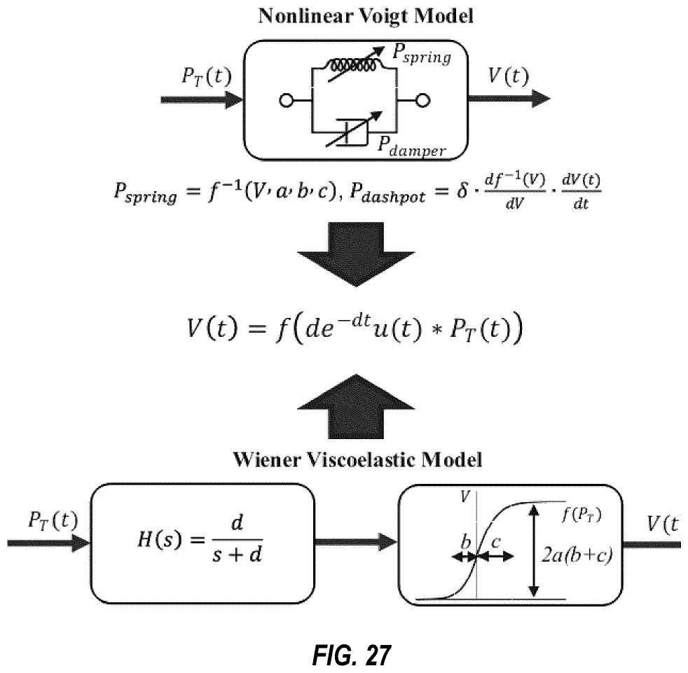

Although the Hammerstein and Wiener models are quite similar in their architecture, which can explain why both models fitted the data similarly, the Wiener model was more representative of finger artery viscoelasticity. This model was better in fitting oscillogram data (FIG. 6B) and in predicting the effect of deconvolution on the oscillogram data (FIGS. 26A-26C). Moreover, the Wiener model has a physical correspondence; that is, it is equivalent to a nonlinear Voigt model of viscoelasticity, as shown in FIG. 27.

The linear Voigt model is the simplest of the physical models of viscoelasticity. It compresses like a purely elastic spring with slow deformation but offers additional resistance to fast deformation via a damper (i.e., it acts as a lowpass filter). The model is a purely viscous dashpot and a purely elastic spring connected in parallel.

The Voigt model was modified for nonlinear arteries. In the modification, pressure was treated as force and volume was treated as length.

First, a nonlinear spring was used, where the reaction pressure applied by the spring is the inverse sigmoidal arterial volume-pressure relationship (see Eqs. (8) and (9)) as follows:

$${P}_{spring} ( t ) = {f}^{- 1} ( V ( t ) , a , b , c ) \qquad ( Eq . 11 )$$

The spring constant or volume elastance can thus be given as follows:

$$k = \frac{{dP}_{spring}}{dV} = \frac{{df}^{- 1} ( V )}{dV} \qquad ( Eq . 12 )$$

Second, a nonlinear dashpot was used. The nonlinearity was defined so that the linearized Voigt model at any arterial volume has a constant transfer function pole (see below for justification). In other words, the damping coefficient 17 increases proportionally with k. The reaction pressure applied by the dashpot is therefore given as follow:

$${P}_{dashpot} ( t ) = η \cdot \frac{dV ( t )}{dt} = δ \cdot k \cdot \frac{dV ( t )}{dt} = δ \cdot \frac{{df}^{- 1} ( V )}{dV} \cdot \frac{dV ( C )}{dV} \qquad ( Eq . 13 )$$

where S is a free parameter of the model.

Putting everything together, the following first-order nonlinear differential equation is obtained:

$${P}_{T} ( t ) = {f}^{- 1} ( V ( t ) ) + δ \cdot \frac{{df}^{- 1} ( V ( t ) )}{dt} \qquad ( Eq . 14 )$$

This equation can be solved for V(t) as follows:

$$V ( t ) = f ( {de}^{- dt} u ( t ) * {P}_{T} ( t ) ) \qquad ( Eq . 15 )$$

where d=1 S, u (t) is again the unit step function, and * is the convolution operation. Eq. 15 is equivalent to the Wiener model with a unity gain single pole transfer function and static sigmoidal nonlinearity (FIG. 27).

The physical correspondence between the Wiener model and nonlinear Voigt model here assumes a constant pole. This assumption can be justified by the ex vivo study of rabbit aortic strips. These investigators used a standard linear solid model of arterial viscoelasticity, wherein the transfer function has one pole and one zero. Their data indicate that the model parameters vary with external force applied to the artery (or pre-stretch). However, the pole of the model transfer function remains constant over the range of stretches. The constant pole is a necessary condition for the Wiener model's physical correspondence to the nonlinear Voigt model.

A zero was added to the transfer function, but it did not improve the model fitting. The different dynamics observed herein can be due to numerous differences such as the tested artery (finger vs aorta), the tested subject (human vs rabbit), or the artery environment (in vivo vs ex vivo).

### Wiener Model Parameter Values

The Wiener model parameters b and c indicate the widths of the arterial compliance curve over the negative and positive transmural pressure regimes, respectively. The b parameter is expected to be small for collapsible finger arteries, whereas the ratio of the b and c parameters can be similar to the brachial artery. The b and c values for the finger arteries in this study were 8.0 (5-10.5) and 12.0 (10.3-16.5) mmHg (FIGS. 6C and 6D). In comparison, the b and c parameter values for the brachial artery in cardiac catheterization patients with stiffer arteries are 11 and 17 mmHg. While the finger artery parameters are smaller than their brachial artery counterparts and the b to c ratios are similar, the b parameter was expected for the finger artery to be even smaller (e.g., <5 mmHg). One potential reason for the higher b parameter values could be inaccuracy of the volume-clamp device in measuring the input finger BP waveform. For instance, if SP were underestimated by the device, the model would have to increase b to compensate. Also note that there was large variability in the b and c parameters (FIGS. 6C and 6D).

The Wiener model transfer function cutoff frequency, which can be readily computed from the d parameter, is inversely related to the extent of finger artery viscoelasticity. The transfer function cutoff frequency value was 3.0 (2.5-4.0) Hz (FIG. 6E). This cutoff frequency is close to the typical heart rates (1-2 Hz). For further comparison, the population average transfer function from brachial to finger BP shows a resonance peak at 7 Hz. This difference additionally underscores the significant impact finger artery viscoelasticity can have on the oscillometric BP computation. Note that the transfer function cutoff frequency is necessarily higher than the cutoff frequency of the highpass filter used to extract P_{PGac}(t). In the end, the overall dynamics relating P_{T}(t) (transmural pressure) to P_{PGac}(t). In the end, the overall dynamics relating P_{T}(t) (blood volume oscillations) exhibit nonlinear bandpass characteristics.

### Wiener Model Simulations

A parametric sensitivity analysis was performed with the Wiener model to illustrate the impact of finger artery viscoelasticity on the derivative algorithm for computing BP (see FIG. 22). The disclosed analysis showed that (1) SP computation via Pₘᵢₙₛₗₒₚₑ is much more affected by viscoelasticity than DP computation via Pₘₐₓₛₗₒₚₑ and (2) PP and the transfer function cutoff frequency are the major contributors to the SP underestimation (FIGS. 7A-7D). This sensitivity analysis is supported by the experimental results where PP tended to be underestimated by the derivative algorithm.

The derivative algorithm was used because it was previously shown that this popular algorithm can yield BP with minimal error for purely elastic, collapsible finger arteries and in the absence of noise. Any significant error of the derivative algorithm in the model simulations would thus be due to viscoelasticity alone. However, these results would apply to other popular oscillometric algorithms, which typically do not take viscoelasticity into account. For instance, the fixed ratio algorithm was applied with 0.85 and 0.55 for the DP and SP ratios, respectively, and found similar results.

The nonlinear Voigt model helps in understanding these results. In this model, the damping reaction pressure is proportional to the damping coefficient and the time rate of change of the artery volume (Eq. 14). Therefore, for a given artery volume elastance and heart rate, large PP would result in large rate of change in artery volume, resulting in a more damped volume waveform. Since damping affects the oscillogram more at high finger pressures due to the sharper volume waveform at these pressures, SP computation is most affected by viscoelasticity. It is important to note that the rate of change in arterial volume can be affected by the shape of the waveform (not only its amplitude, i.e., PP), which was kept constant in the simulations. Heart rate was also kept constant in the simulations but will have a similar effect on the damping reaction pressure. For instance, increased heart rate will proportionally increase the damping leading to the same effect as PP on BP computation algorithms. A particularly important feature for the underestimation of SP can be the finger BP upstroke time. The sensitivity analysis was kept simple by using only PP to express the shape of the input to the model.

### Potential Remedies to Compensate for the Adverse Effects of Finger Artery Viscoelasticity

Potential remedies to compensate for the adverse effects of finger artery viscoelasticity in the BP computation were explored. The PPG_{ac} waveform shape can provide information about the system characteristics. More precisely, it can provide an estimation of the cutoff frequency of the transfer function (FIGS. 8A-8C). It is also shown that PPGₘₑₐₙ—the average of the normalized PPG_{ac} waveform at the maximum oscillation beat-correlates well with the transfer function cutoff frequency (r=−0.8). Although the mean value was employed here, any metric representing bulkiness could be used including the root-mean-square (r=−0.83, not shown). These markers could be used as input to an algorithm to compensate for BP computation error caused by viscoelasticity. For instance, they could be used directly as input (along with other features extracted from the oscillometric finger pressing data) to a machine learning model to compute SP and DP. Another example is to use the model to compensate for the error in popular algorithms (e.g., derivative algorithm). For instance, for a given algorithm, a mapping from PP estimate and cutoff frequency to BP error (FIGS. 7A-7D) can be created using training data comprising oscillometric finger pressing data and reference cuff BP. Then, for a given finger pressing measurement, PP is computed (e.g., via the derivative algorithm), the cutoff frequency is estimated (e.g., via PPGₘₑₐₙ), and the BP error can thus be calculated via the aforementioned mapping.

Deconvolution was used herein as a method to distinguish the Wiener model from the Hammerstein model and show that actual finger pressing data display similar behavior to the Wiener model (FIG. 26A-26C). Even though deconvolution of the PPG_{ac} waveform cannot be able to remove viscoelastic effects from the oscillogram, it can still provide an estimate of the BP waveform shape. In fact, deconvolving the PPG_{ac} waveform beat at maximum oscillation with the estimated transfer function can provide an accurate BP waveform shape, since the sigmoidal function is approximately linear at around the maximum oscillation. This beat could be used in advanced algorithms to compute BP. For instance, features from the derived beat could be used as input to a machine learning model to compute BP. Alternatively, an absolute BP waveform beat can be determined using deconvolution. First, a popular algorithm is applied to compute SP and DP. Then, the PPG_{ac} waveform beat at maximum oscillation is calibrated to these BP levels. Next, the transfer function with estimated cutoff frequency is deconvolved from the beat. Finally, the maximum and minimum of the deconvolved beat are detected to arrive at SP and DP corrected for finger artery viscoelasticity.

A significant weakness of pure PPG markers of viscoelasticity is that the bulkiness of the normalized PPG_{ac} waveform at the maximum oscillation beat reflects both the transfer function cutoff frequency and the input finger BP waveform. Therefore, a change in the BP waveform (due to, e.g., aging) can erroneously be interpreted as a change in the system viscoelasticity. Nevertheless, here, viscoelasticity was a prominent contributor to PPGₘₑₐₙ. It is also possible that viscoelastic effects are more important in determining PPG bulkiness in general.

Whether finger artery viscoelasticity needs to be taken into account in the context of oscillometric finger pressing method for potential cuffless BP monitoring was assessed, and if so, how viscoelasticity affects the oscillometric BP computation was assessed. Nonlinear viscoelastic models and fitted models were developed for oscillometric finger-pressing data. A Wiener viscoelastic model, consisting of a first-order linear transfer function followed by a static sigmoidal function, fitted the data much better than a purely elastic model, thereby indicating significant effect of viscoelasticity in oscillometric finger pressing. The Wiener model explained the data better than a Hammerstein viscoelastic model and carries physical meaning. Through simulations with the Wiener model, it is found that viscoelasticity leads to mild misestimation of DP but large underestimation of SP when using a popular oscillometric BP computation algorithm. The SP underestimation increases with both PP and the inverse of the transfer function cutoff frequency, which is an index of arterial viscoelasticity. Although viscoelasticity negatively impacts BP computation algorithms, it is discovered that PPG waveform markers of the transfer function cutoff frequency, which could potentially be used for enhancing BP computation accuracy without additional sensors. The disclosed subject matter can improve the understanding of finger artery viscoelasticity in the context of the oscillometric finger pressing method and can be an important step toward improving hypertension awareness and control via smartphone-based BP monitoring.

### Example 2: Oscillometric Blood Pressure Measurement: Mathematical Modeling of Area and Height Oscillograms

Here, mathematical models of oscillometry were developed to relate cuff pressure oscillation area and height to cuff pressure. The assumptions of the models were tested, and the models were able to predict measurements well (N=128). The disclosed models can help in improving the accuracy of popular oscillometric blood pressure measurements.

Oscillometry has become an important principle for measuring blood pressure (BP). It involves the assessment of cuff pressure oscillations relative to the cuff pressure applied to an artery. Traditional oscillometry relies solely on oscillation height variations versus cuff pressure (‘oscillogram’) for BP estimation. However, the overall shape of the oscillations also changes. Analytical models of the area and height oscillograms were developed. Both models assume that the brachial artery and cuff are purely elastic and that a constant scale factor links blood volume to cuff pressure oscillations. The area oscillogram model further assumes that the arterial BP waveform shape is triangular. Here, the validity of these assumptions in terms of model fitting to measured oscillograms were assessed.

The mathematical models of the height and area oscillograms (H(P_{c}) and A(P_{c})) are as follows:

$$H ( {P}_{c} ) = k [ f ( {P}_{s} - {P}_{c} , a , b , c ) - f ( {P}_{d} - {P}_{c} , a , b , c ) ] \qquad ( Eq . 8 )$$

$$A ( {P}_{c} ) = k {\int }_{0}^{T} ( f ( {P}_{a} ( T ) - {P}_{c} , a , b , c ) - f ( {P}_{d} - {P}_{c} ) ) dt , \qquad ( Eq . 9 )$$

where P_{c} is cuff pressure, Pₐ(t) is the BP waveform with systolic and diastolic BP (Pₛ and P_{d}), ƒ(·) is the sigmoidal arterial blood volume-transmural pressure relationship defined by an exponential-linear function with parameters a, b, c, T is the beat length, and k is a scale factor relating blood volume to cuff pressure oscillations. To derive an analytical expression for A(P_{c}), Pₐ(t) is modeled as a triangle.

The disclosed models were assessed using oscillometric arm cuff pressure waveforms and invasive brachial BP waveforms from 128 cardiac catheterization patients. Then, area and height oscillograms were formed. Both models were inputted with the cuff pressure waveform and invasive systolic and diastolic BP and performed least squares optimization to fit the model predicted oscillograms to the measured oscillograms.

The validity of the model assumptions was assessed. The first assumption is that a triangular pulse can represent the BP waveform shape. The invasive brachial BP waveform was used as input and then performed the model fitting. The second assumption posits that the cuff-artery system is purely elastic and thus disregards any viscoelastic properties. Viscoelastic Wiener and Hammerstein models, which include a linear damper H(s)=d/(s+d) and static nonlinearity (ƒ(·)), were used, and then the model fitting was performed. The third assumption is that a constant scale factor k couples the blood volume and cuff pressure oscillations and thus ignores potential nonlinearities or effects due to air compressibility within the cuff. Utilizing the cuff-artery model, the relationship between blood volume and cuff pressure oscillations in the linear region of the cuff is k_{eff}=[P_{c}/P_{A}+1]k₁, where P_{A} is atmospheric pressure and k₁ is the linear elasticity of the cuff. k_{eff} was used for the scaling and then performed the model fitting.

FIGS. 15C-15E illustrate the results in which each assumption was tested individually. The triangular pulse input actually yielded better fitting of the area oscillograms than the invasive BP waveform input (FIG. 15C). The Wiener and Hammerstein models fitted the height oscillograms better than the purely elastic model (FIG. 15D). In contrast, the area oscillogram model proved notably more resilient to viscoelastic effects (FIG. 15D). The nonconstant scale factor did not yield significantly better fitting of the area or height oscillograms (FIG. 15E). Testing the three assumptions collectively did not change the results.

The findings suggest that both the area and height oscillogram models can fit measurements. The area oscillogram model appears more robust to the model assumptions than the height oscillogram model. The disclosed subject matter can ultimately help in improving the accuracy of popular oscillometric BP measurements.

### Example 3: A Smartphone-Based Device to Measure Venous Pressure

An oscillometric finger pressing device was built to conveniently measure venous pressure. The disclosed device can track increases in venous pressure in healthy volunteers (N=15). The disclosed device in a smartphone form factor could help reduce fluid overload hospitalizations.

Fluid overload causes frequent hospitalizations in congestive heart failure patients and preeclamptic individuals postpartum. If the fluid overload could be predicted beforehand, then diuretic therapy could be given to avert the hospitalization. Rising heart pressure is the cause of fluid overload and occurs well before symptom onset. However, heart pressure is nominally measured invasively. Here, a smartphone-based device was proposed to measure finger venous pressure (VP) as a surrogate of right heart pressure.

The disclosed device employs the oscillometric principle, which is employed by most automatic cuff devices to measure arterial pressure. This principle can also measure VP via a peak in the cuff pressure oscillation amplitude versus cuff pressure function (“oscillogram”) near the VP range (e.g., 5-20 mmHg). However, the low cuff pressure needed to detect VP often results in poor contact between the cuff and arm, leading to noisy readings. To improve the signal-to-noise ratio and realize a smartphone form factor, the oscillometric finger pressing method was utilized. The idea is for the user to slowly press their fingertip against a photoplethysmography (PPG)-force sensor unit via visual guidance while holding the device well below the heart to increase the finger VP by the hydrostatic pressure. The hydrostatic pressure is known from the user arm length and subtracted from the oscillometric measurement to yield VP using higher external pressures.

To test the idea, a benchtop device was used to measure fingertip PPG and pressure during visually-guided oscillometric finger pressing. Initial human studies were conducted in 15 volunteers. Each study participant was seated in a height-adjustable chair, wore a manual arm cuff, and had their hands fully lowered (FIG. 20A). The participant performed the oscillometric finger pressing method with the arm cuff inflated to 0, 25, and 50 mmHg to increase the VP in the finger via venous pooling.

An example of the oscillometric measurements is shown (FIG. 20B). The maximum VP oscillations (mean VP) is at about 24, 31, and 54 mmHg with increasing arm cuff pressure.

The VP is shown for each study participant at the different arm cuff pressures (FIG. 20D). The finger VP is nominally around 21 mmHg and then increases with arm cuff pressure.

This line of research can lead to a ubiquitous device to reduce fluid overload hospitalizations especially in the younger postpartum population.

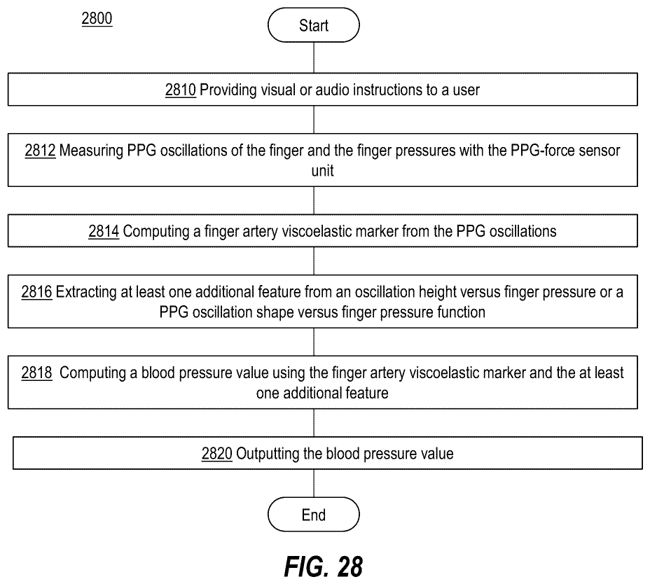

Various embodiments disclosed herein describe methods and systems for measuring a user's blood pressure. An illustrative method 2800 for measuring blood pressure is shown in FIG. 28. Method 2800 can include providing visual or audio instructions to a user at step 2810. In non-limiting embodiments, the user can utilize any suitable electronic device capable of providing visual and/or audio instructions. These instructions can explain how to use a photoplethysmography (PPG)-force sensor unit to obtain data for determining the user's blood pressure.

In various cases, at step 2812 method 2800 can include measuring finger PPG oscillations and finger pressure. Visual and/or audio instructions can guide the user to position a finger on the PPG-force sensor unit and apply varying finger pressures. Upon pressing the finger, at step 2812, the sensor unit measures both PPG oscillations and the pressure from the finger.

Further, method 2800 can include at step 2814 computing a finger artery viscoelastic marker from the PPG oscillations. In non-limiting embodiments, a viscoelastic marker may include an average of a normalized maximum PPG oscillation waveform, a root-mean-square of the normalized maximum PPG oscillation waveform, or other relevant metric.

At step 2816, method 2800 can include extracting at least one additional feature from a PPG oscillation height versus finger pressure or a PPG oscillation shape versus finger pressure function. Such an additional feature can include a width of the oscillation vs finger pressure function, an area of the oscillation vs finger pressure function, or another shape of the oscillation vs finger pressure function.

At step 2818, method 2800 can include computing a blood pressure value, using the finger artery viscoelastic marker along with at least one additional feature. Further, after computing a blood pressure value at step 2818, method 2800 can include, at step 2820 outputting the computed blood pressure value to a suitable display, or via any other media (e.g., via an audio signal or a printed paper copy).

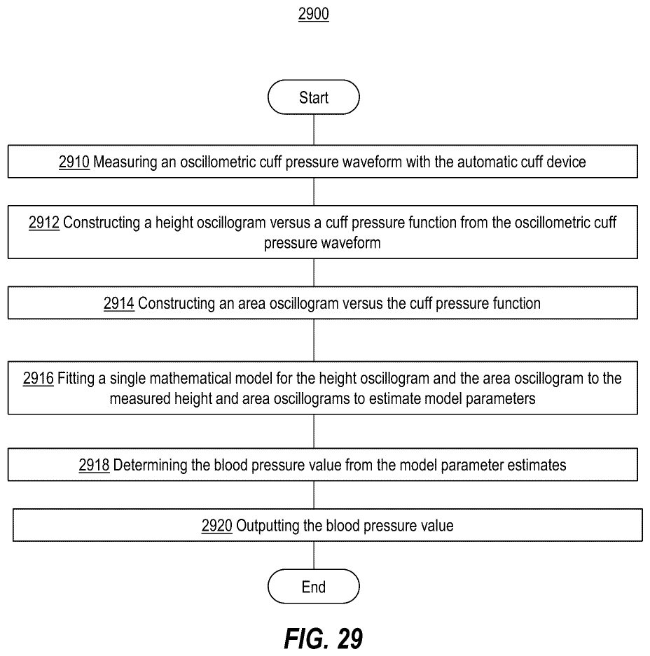

Another example method 2900 of determining a blood pressure value is shown in FIG. 29. Method 2900 can include at step 2910, measuring an oscillometric cuff pressure waveform with the automatic cuff device. The cuff can be any suitable device configured to measure arterial oscillations and the external pressure applied to the artery.

At step 2912, method 2900 can include constructing a height oscillogram obtained from the oscillometric cuff pressure waveform as the oscillation height versus cuff pressure function. At step 2914, method 2900 can include determining an area oscillogram from the oscillometric cuff pressure waveform as the oscillation area versus cuff pressure function. At step 2916, method 2900 can include fitting a single mathematical model for the height and the area oscillograms to measured oscillograms in an optimal sense to estimate model parameters, wherein parameters of the single mathematical model include systolic blood pressure, diastolic blood pressure, and arterial compliance. At step 2918, method 2900 can optionally include determining or selecting a blood pressure value from the model parameter estimates. Further, at step 2920, method 2900 can include outputting the blood pressure value to a suitable display, or via any other media. In non-limiting embodiments, the model can be fitted over a higher cuff pressure range. In non-limiting embodiments, the pressure-volume relation of a cuff transducer is more linear than a pressure-volume relation obtained at a lower cuff pressure range. For example, the pressure-volume relation of the cuff is a curve that is concave upward. Its local derivative increases with cuff pressure. When cuff pressure is higher, it is more linear (i.e., the local derivative is more constant).

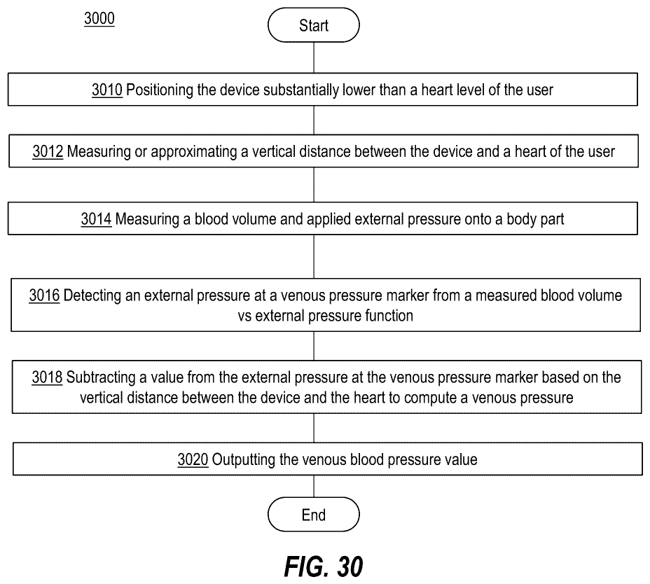

FIG. 30 shows an example method 3000 for obtaining a venous blood pressure value for a user using an oscillometric device. Method 3000 can include, at step 3010, positioning a device for measuring blood pressure substantially lower than the heart level of the user, and at step 3012, measuring or approximating the vertical distance between the device and the user's heart. Further, at step 3014, method 3000 can include measuring a blood volume that can correspond to venous blood volume. Additionally, at step 3014, the applied external pressures on a vein can be measured. At step 3016, method 3000 can include detecting an external pressure at a venous pressure marker from a measured blood volume vs external pressure function. At step 3018, method 3000 can include subtracting a value from the external pressure at the venous pressure marker based on the vertical distance between the device and the heart to compute a venous pressure. This value can be, for example, the hydrostatic pressure. At step 2620, method 2600 can include outputting the venous blood pressure value to a suitable device (e.g., a smartphone, a monitor, or a printer).

### REFERENCES

The following references disclose subject matter that is incorporated by reference herein in their entireties:
ADDIN ZOTERO_BIBL {“uncited”: [ ], “omitted”: [ ], “custom”: [ ]} CSL_BIBLIOGRAPHY
[1] A. Chandrasekhar, C.-S. Kim, M. Naji, K. Natarajan, J.-O. Hahn, and R. Mukkamala, “Smartphone-based blood pressure monitoring via the oscillometric finger-pressing method,” Sci. Transl. Med., vol. 10, no. 431, p. caap8674, March 2018, doi: 10.1126/scitranslmed.aap8674.
[2] A. Chandrasekhar et al., “Formulas to Explain Popular Oscillometric Blood Pressure Estimation Algorithms,” Front. Physiol., vol. 10, 2019, Accessed: Feb. 23, 2022. [Online]. Available: https://www.frontiersin.org/article/10.3389/fphys.2019.01415
[3] M. Freithaler, A. Chandrasekhar, V. Dhamotharan, C. Landry, S. G. Shroff, and R. Mukkamala, “Smartphone-Based Blood Pressure Monitoring via the Oscillometric Finger Pressing Method: Analysis of Oscillation Width Variations Can Improve Diastolic Pressure Computation,” IEEE Trans. Biomed. Eng., pp. 1-11, 2023, doi: 10.1109/TBME.2023.3275031.
[4] G. J. Langewouters, A. Zwart, R. Busse, and K. H. Wesseling, “Pressure-diameter relationships of segments of human finger arteries,” Clin. Phys. Physiol. Meas., vol. 7, no. 1, p. 43, February 1986, doi: 10.1088/0143-0815/7/1/003.
[5] J. Liu et al., “Patient-Specific Oscillometric Blood Pressure Measurement,” IEEE Trans. Biomed. Eng., vol. 63, no. 6, pp. 1220-1228 June 2016, doi: 10.1109/TBME.2015.2491270.
[6] V. Dhamotharan et al., “Mathematical Modeling of Oscillometric Blood Pressure Measurement: A Complete, Reduced Oscillogram Model,” IEEE Trans. Biomed. Eng., pp. 1-8, 2022, doi: 10.1109/TBME.2022.3201433.
[7] H.-M. Cheng, S.-H. Sung, Y.-T. Shih, S.-Y. Chuang, W.-C. Yu, and C.-H. Chen, “Measurement of Central Aortic Pulse Pressure: Noninvasive Brachial Cuff-Based Estimation by a Transfer Function Vs. a Novel Pulse Wave Analysis Method,” Am. J. Hypertens., vol. 25, no. 11, pp. 1162-1169 November 2012, doi: 10.1038/ajh.2012.116.
[8] H.-M. Cheng, S.-H. Sung, Y.-T. Shih, S.-Y. Chuang, W.-C. Yu, and C.-H. Chen, “Measurement Accuracy of a Stand-Alone Oscillometric Central Blood Pressure Monitor: A Validation Report for Microlife WatchBP Office Central,” Am. J. Hypertens., vol. 26, no. 1, pp. 42-50, January 2013, doi: 10.1093/ajh/hps021.
[9] J. Liu, H.-M. Cheng, C.-H. Chen, S.-H. Sung, J.-O. Hahn, and R. Mukkamala, “Patient-Specific Oscillometric Blood Pressure Measurement: Validation for Accuracy and Repeatability,” IEEE J. Transl. Eng. Health Med., vol. 5, pp. 1-10, 2017, doi: 10.1109/JTEHM.2016.2639481.
[10] T. Hidaka et al., “Non-Invasive Central Venous Pressure Measurement Using Enclosed-Zone Central Venous Pressure (ezCVP™),” Circ. J., vol. 84, no. 7, pp. 1112-1117 June 2020, doi: 10.1253/circj.CJ-20-0129.
[11] B. Zhou et al., “Worldwide Trends in Hypertension Prevalence and Progress in Treatment and Control from 1990 to 2019: A Pooled Analysis of 1201 Population-Representative Studies with 104 Million Participants,” The Lancet, vol. 398, no. 10304, pp. 957-980, September 2021, doi: 10.1016/S0140-6736 (21) 01330-1.
[12] R. Mukkamala, G. S. Stergiou, and A. P. Avolio, “Cuffless Blood Pressure Measurement,” Annu. Rev. Biomed. Eng., vol. 24, no. 1, pp. 203-230, 2022, doi: 10.1146/annurev-bioeng-110220-014644.
[13] C. Landry, V. Dhamotharan, M. Freithaler, A. Hauspurg, M. F. Muldoon, S. G. Shroff, A. Chandrasekhar, and R. Mukkamala, “A Smartphone Application Toward Detection of Systolic Hypertension in Underserved Populations,” Sci Rep, vol. 14, no. 1, p. 15410, 2024.
[14] I. W. Hunter and M. J. Korenberg, “The Identification of Nonlinear Biological Systems: Wiener and Hammerstein Cascade Models,” Biol. Cybern., vol. 55, no. 2, pp. 135-144, November 1986, doi: 10.1007/BF00341929.
[15] L. A. Aguirre, M. C. S. Coelho, and M. V. Corrêa, “On the interpretation and practice of dynamical differences between Hammerstein and Wiener models,” IEE Proc.-Control Theory Appl., vol. 152, no. 4, pp. 349-356, July 2005, doi: 10.1049/ip-cta: 20045152.
[16] R. H. Byrd, J. C. Gilbert, and J. Nocedal, “A Trust Region Method Based on Interior Point Techniques for Nonlinear Programming,” Math. Program., vol. 89, no. 1, pp. 149-185, November 2000, doi: 10.1007/PL00011391.
[17] R. Mukkamala, J.-O. Hahn, and A. Chandrasekhar, “11-Photoplethysmography in Noninvasive Blood Pressure Monitoring,” in Photoplethysmography, J. Allen and P. Kyriacou, Eds., Academic Press, 2022, pp. 359-400. doi: 10.1016/B978-O-12-823374-0.00010-4.
[18] C. F. Babbs, “Oscillometric Measurement of Systolic and Diastolic Blood Pressures Validated in a Physiologic Mathematical Model,” Biomed. Eng. OnLine, vol. 11, no. 1, p. 56, August 2012, doi: 10.1186/1475-925X-11-56.
[19] M. Wurzel, G. R. Cowper, and J. M. Mc Cook, “Smooth Muscle Contraction and Viscoelasticity of Arterial Wall,” Can. J. Physiol. Pharmacol., vol. 48, no. 8, pp. 510-523, August 1970, doi: 10.1139/y70-079.
[20] P. Gizdulich and K. H. Wasseling, “Reconstruction of Brachial Arterial Pulsation From Finger Arterial Pressure,” in Proceedings of the Twelfth Annual International Conference of the IEEE Engineering in Medicine and Biology Society, November 1990, pp. 1046-1047. doi: 10.1109/IEMBS.1990.691590.
[21] G. Drzewiecki, R. Hood, and H. Applet, “Theory of the Oscillometric Maximum and the Systolic and Diastolic Detection Ratios,” Annals of Biomedical Engineering, vol. 22, pp. 88-96, 1994

What is claimed is:

1. A method for determining a blood pressure value of a user using a device with a photoplethysmography (PPG)-force sensor unit comprising:
providing visual or audio instructions to the user with the device, wherein the instructions instruct the user to position a finger on the PPG-force sensor unit and to press the finger on the PPG-force sensor unit at varying finger pressures;
measuring PPG oscillations of the finger and the finger pressures with the PPG-force sensor unit;
computing a finger artery viscoelastic marker from the PPG oscillations;
extracting at least one additional feature from an oscillation height versus finger pressure or a PPG oscillation shape versus finger pressure function;
computing a blood pressure value using the finger artery viscoelastic marker and the at least one additional feature; and
outputting the blood pressure value on a graphical user interface of the device or sending the blood pressure value to a database repository.

2. The method of claim 1, wherein the finger artery viscoelastic marker comprises an average of a normalized PPG waveform.

3. The method of claim 1, wherein the finger artery viscoelastic marker comprises a root-mean-square of a normalized PPG waveform.

4. The method of claim 1, wherein the at least one additional feature comprises a width or peak position of the height oscillation vs finger pressure function.

5. The method of claim 1, wherein the at least one additional feature is extracted from an oscillation width vs finger pressure function.

6. A method for determining a blood pressure value of a user using an automatic cuff device comprising:
measuring an oscillometric cuff pressure waveform with the automatic cuff device;
constructing a height oscillation versus a cuff pressure function from the oscillometric cuff pressure waveform;
constructing an area oscillation versus the cuff pressure function of the oscillometric cuff pressure waveform;
fitting a single mathematical model for the height and the area functions to the measured height oscillation versus the cuff pressure function and the measured area oscillation versus the cuff pressure function, respectively, in an optimal sense to estimate model parameters, wherein parameters of the single mathematical model include systolic blood pressure, diastolic blood pressure, and arterial compliance; and
outputting a blood pressure value on a graphical user interface of the device or sending the blood pressure value to a database repository.

7. The method of claim 6, wherein the mathematical model is a sigmoidal blood volume-transmural pressure relation of arteries.

8. The method of claim 7, wherein an arterial compliance curve, which is a derivative of the sigmoidal relation, is defined by an asymmetric exponential function.

9. The method of claim 7, wherein an arterial compliance curve, which is a derivative of the sigmoidal relation, is defined by an asymmetric exponential-linear function.

10. The method of claim 6, wherein the mathematical model of the area function uses a triangular pulse train to represent a blood pressure waveform input.

11. The method of claim 6, wherein the model is fitted over a higher cuff pressure range, wherein a pressure-volume relation of a cuff transducer is more linear than a pressure-volume relation obtained at a lower cuff pressure range.

12. A method for determining a venous pressure value of a user using an oscillometric device on a wrist or a hand comprising:
positioning the device lower than a heart level of the user;
measuring or approximating a vertical distance between the device and a heart of the user;
measuring a blood volume and an applied external pressure of a vein at varying external pressure;
detecting an external pressure at a venous pressure marker from a measured blood volume vs external pressure function;
subtracting a value from the external pressure at the venous pressure marker based on the vertical distance between the device and the heart to compute a venous pressure; and
outputting the venous pressure value on a graphical user interface of the device or sending the venous pressure value to a database repository.

13. The method of claim 12, wherein the device is an automatic wrist cuff device.

14. The method of claim 12, wherein the device is an oscillometric finger pressing device, wherein the device is held with the hand fully lowered.

15. The method of claim 12, wherein the venous pressure is detected from a peak position in blood volume oscillation vs external pressure function that is lower than arterial pressure levels.

16. The method of claim 12, wherein the venous pressure is detected from a DC PPG measurement of the blood volume.

17. A system for determining a blood pressure value of a user, comprising
a photoplethysmography (PPG)-force sensor, and
a processor configured to
provide visual or audio instructions to the user with the system, wherein the instructions instruct the user to position a finger on a PPG-force sensor unit and to press the finger on a PPG-force sensor unit at varying finger pressures;
measure PPG oscillations of the finger and the finger pressures with the PPG-force sensor unit;
compute a finger artery viscoelastic marker from the PPG oscillations;
extract at least one additional feature from a PPG oscillation height versus finger pressure or a PPG oscillation shape versus finger pressure function;
compute a blood pressure value using the finger artery viscoelastic marker and the at least one additional feature; and
output the blood pressure value on a graphical user interface of the system or send the blood pressure value to a database repository.

18. An automatic cuff system for determining a blood pressure value of a user comprising:
a processor configured to:
measure an oscillometric cuff pressure waveform with the automatic cuff device;
construct a height oscillogram versus a cuff pressure function from the oscillometric cuff pressure waveform;
construct an area oscillogram versus the cuff pressure function of the oscillometric cuff pressure waveform;
fit a single mathematical model for the height oscillogram and the area oscillogram to measured height and area oscillograms in an optimal sense to estimate model parameters, wherein parameters of the single mathematical model include systolic blood pressure, diastolic blood pressure, and arterial compliance; and
output a blood pressure value on a graphical user interface of the device or send the blood pressure value to a database repository.

19. A system for determining a venous pressure value of a user; comprising:
an oscillometric device on a wrist or a hand; and
a processor configured to:
provide visual or audio instructions to the user to position the device lower than a heart level of the user;
measure or approximate a vertical distance between the device and a heart of the user;
measure a blood volume and an applied external pressure onto the wrist or hand at varying external pressure;
detect an external pressure at a venous pressure marker from a measured blood volume vs external pressure function;
subtract a value from the external pressure at the venous pressure marker based on the vertical distance between the device and the heart to compute a venous pressure; and
output the venous pressure value on a graphical user interface of the device or send the venous pressure value to a database repository.

20. The system of claim 17, wherein the finger artery viscoelastic marker comprises at least one of an average of a normalized PPG waveform and a root-mean-square of a normalized PPG waveform.
